(19) United States
(12) Patent Application Publication
(10) Pub. No.: US 2017/0049317 A1
(43) Pub. Date: Feb. 23, 2017

(54) MEMBRANE VISUALIZATION INSTRUMENT

(71) Applicant: Katalyst Surgical, LLC, Chesterfield, MO (US)

(72) Inventors: Gregg D. Scheller, Wildwood, MO (US); Nancy M. Holekamp, St. Louis, MO (US)

(73) Assignee: Katalyst Surgical, LLC, Chesterfield, MO (US)

(21) Appl. No.: 15/342,871

(22) Filed: Nov. 3, 2016

Related U.S. Application Data
(63) Continuation of application No. 14/100,979, filed on Dec. 9, 2013.

(51) Int. Cl. A61B 3/10; A61F 9/007

(52) U.S. Cl. CPC A61B 3/10; A61F 9/00736

(57) ABSTRACT

A membrane visualization instrument may include a flow control mechanism having a flow control mechanism distal end and a flow control mechanism proximal end, a visualization fluid chamber of the flow control mechanism, a visualization fluid guide having a visualization fluid guide distal end and a visualization fluid guide proximal end, and a hypodermic tube having a hypodermic tube distal end and a hypodermic tube proximal end. The visualization fluid guide proximal end may be disposed within the flow control mechanism. The hypodermic tube proximal end may be disposed within the visualization fluid guide. A visualization fluid, e.g., indocyanine green dye, kenalog, trypan blue dye, etc., may be disposed within the visualization fluid chamber. A compression of the flow control mechanism may be configured to irrigate the visualization fluid out of the hypodermic tube proximal end.

### CROSS-REFERENCE TO RELATED APPLICATIONS

This application is a continuation of prior application Ser. No. 14/100,979, filed Dec. 9, 2013.

### FIELD OF THE INVENTION

The present disclosure relates to a medical device, and, more particularly, to a surgical instrument.

### BACKGROUND OF THE INVENTION

Some ophthalmic surgical procedures require identification and removal of membranes. An internal limiting membrane is the structural interface between the retina and the vitreous. Ophthalmic surgeons remove the internal limiting membrane to treat a variety of retinal disorders, e.g., macular hole, epiretinal membrane, diabetic macular edema, retinal vein occlusion, etc. The removal of the internal limiting membrane is typically accomplished by incising and peeling the internal limiting membrane from the retina. Removing the internal limiting membrane is difficult because the internal limiting membrane is transparent and invisible under a microscope. Accordingly, there is a need for an instrument for internal limiting membrane visualization and removal.

Epiretinal membranes are thin sheets of fibrous tissue that may form in the eye. Ophthalmic surgeons remove epiretinal membranes to treat a variety of ophthalmic conditions, e.g., metamorphopsia. Unfortunately, epiretinal membranes are often transparent and difficult to identify. For example, it may be difficult for a surgeon to visualize all or a portion of an epiretinal membrane. Accordingly, there is a need for an instrument for epiretinal membrane visualization and removal.

### BRIEF SUMMARY OF THE INVENTION

The present disclosure presents a membrane visualization instrument. Illustratively, a membrane visualization instrument may comprise a flow control mechanism having a flow control mechanism distal end and a flow control mechanism proximal end, a visualization fluid chamber of the flow control mechanism, a visualization fluid guide having a visualization fluid guide distal end and a visualization fluid guide proximal end, and a hypodermic tube having a hypodermic tube distal end and a hypodermic tube proximal end. In one or more embodiments, the visualization fluid guide proximal end may be disposed within the flow control mechanism. Illustratively, the hypodermic tube proximal end may be disposed within the visualization fluid guide. In one or more embodiments, a visualization fluid, e.g., indocyanine green dye, kenalog, trypan blue dye, etc., may be disposed within the visualization fluid chamber. Illustratively, a compression of the flow control mechanism may be configured to irrigate the visualization fluid out of the hypodermic tube proximal end. In one or more embodiments, a decompression of the flow control mechanism may be configured to aspirate the visualization fluid into the hypodermic tube proximal end.

### BRIEF DESCRIPTION OF THE DRAWINGS

The above and further advantages of the present invention may be better understood by referring to the following description in conjunction with the accompanying drawings in which like reference numerals indicate identical or functionally similar elements:

FIGS. 1A and 1B are schematic diagrams illustrating a flow control mechanism;

FIG. 2 is a schematic diagram illustrating an exploded view of a membrane visualization instrument assembly;

FIGS. 3A and 3B are schematic diagrams illustrating an assembled membrane visualization instrument;

FIGS. 4A, 4B, and 4C are schematic diagrams illustrating an application of a visualization fluid to an internal limiting membrane;

FIGS. 5A, 5B, and 5C are schematic diagrams illustrating a removal of a visualization fluid from a surgical site;

FIGS. 6A and 6B are schematic diagrams illustrating a flow control mechanism;

FIG. 7 is a schematic diagram illustrating an exploded view of a membrane visualization instrument assembly;

FIGS. 8A and 8B are schematic diagrams illustrating an assembled membrane visualization instrument;

FIGS. 9A, 9B, and 9C are schematic diagrams illustrating an application of a visualization fluid to an internal limiting membrane;

FIGS. 10A, 10B, and 10C are schematic diagrams illustrating a removal of a visualization fluid from a surgical site;

FIG. 11 is a schematic diagram illustrating a membrane visualization instrument with a blunt tip;

FIG. 12 is a schematic diagram illustrating a membrane visualization instrument with a soft tip;

FIG. 13 is a schematic diagram illustrating a membrane visualization instrument for removing membranes.

### DETAILED DESCRIPTION OF AN ILLUSTRATIVE EMBODIMENT

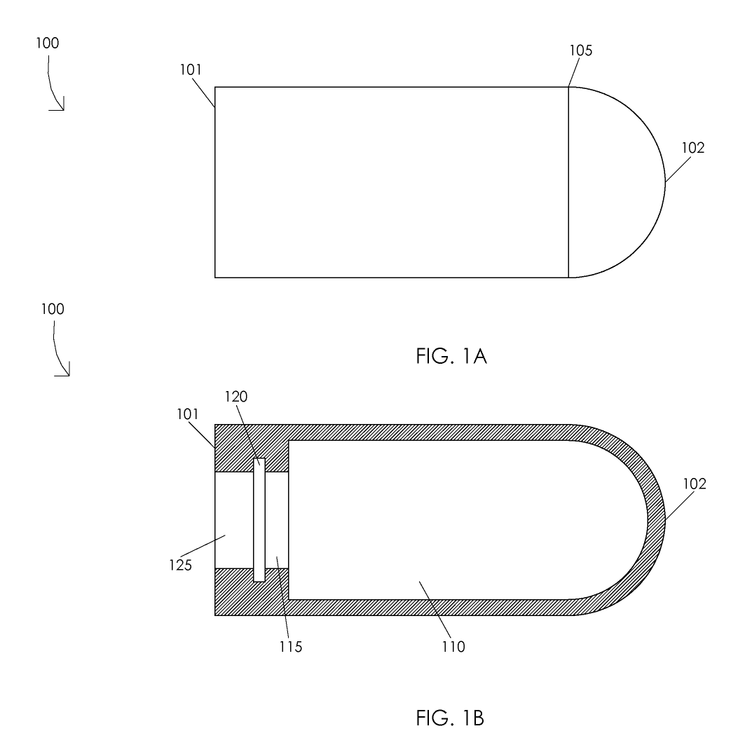

FIGS. 1A and 1B are schematic diagrams illustrating a flow control mechanism 100. FIG. 1A illustrates a top view of a flow control mechanism 100. Illustratively, flow control mechanism 100 may comprise a flow control mechanism distal end 101, a flow control mechanism proximal end 102, and a flow control mechanism dome interface 105. In one or more embodiments, flow control mechanism 100 may be manufactured from any suitable material, e.g., polymers, metals, metal alloys, etc., or from any combination of suitable materials.

FIG. 1B illustrates a cross-sectional view of a flow control mechanism 100. Illustratively, flow control mechanism 100 may comprise a visualization fluid chamber 110, a locking depression housing 115, a distal locking lip housing 120, and a visualization fluid guide housing 125. In one or more embodiments, flow control mechanism 100 may have a density in a range of 0.02 to 0.06 pounds per cubic inch, e.g., flow control mechanism 100 may have a density of 0.0399 pounds per cubic inch. Illustratively, flow control mechanism 100 may have a density less than 0.02 pounds per cubic inch or greater than 0.06 pounds per cubic inch. In one or more embodiments, flow control mechanism 100 may have a mass in a range of 0.003 to 0.007 pounds, e.g., flow control mechanism 100 may have a mass of 0.005 pounds. Illustratively, flow control mechanism 100 may have a mass less than 0.003 pounds or greater than 0.007 pounds. In one or more embodiments, flow control mechanism 100 may have a volume in a range of 0.06 to 0.18 cubic inches, e.g., flow control mechanism 100 may have a volume of 0.126 cubic inches. Illustratively, flow control mechanism 100 may have a volume less than 0.06 cubic inches or greater than 0.18 cubic inches. In one or more embodiments, flow control mechanism 100 may have a surface area in a range of 4.4 to 4.8 square inches, e.g., flow control mechanism 100 may have a surface area of 4.6 square inches. Illustratively, flow control mechanism 100 may have a surface area less than 4.4 square inches or greater than 4.8 square inches.

In one or more embodiments, flow control mechanism 100 may be manufactured from a material suitable for sterilization by a medical autoclave. Illustratively, flow control mechanism 100 may be manufactured from a material, e.g., rubber, configured to withstand exposure to temperatures, pressures, and ambient conditions present in a medical autoclave without degradation. For example, flow control mechanism 100 may be configured to function normally after exposure in a temperature 250° F. for 15 minutes at an atmospheric pressure of 15 psi. In one or more embodiments, flow control mechanism 100 may be configured to be used in a surgical procedure and then sterilized by a medical autoclave at least three times. Illustratively, flow control mechanism 100 may be configured to be used in a surgical procedure and then sterilized by a medical autoclave more than three times.

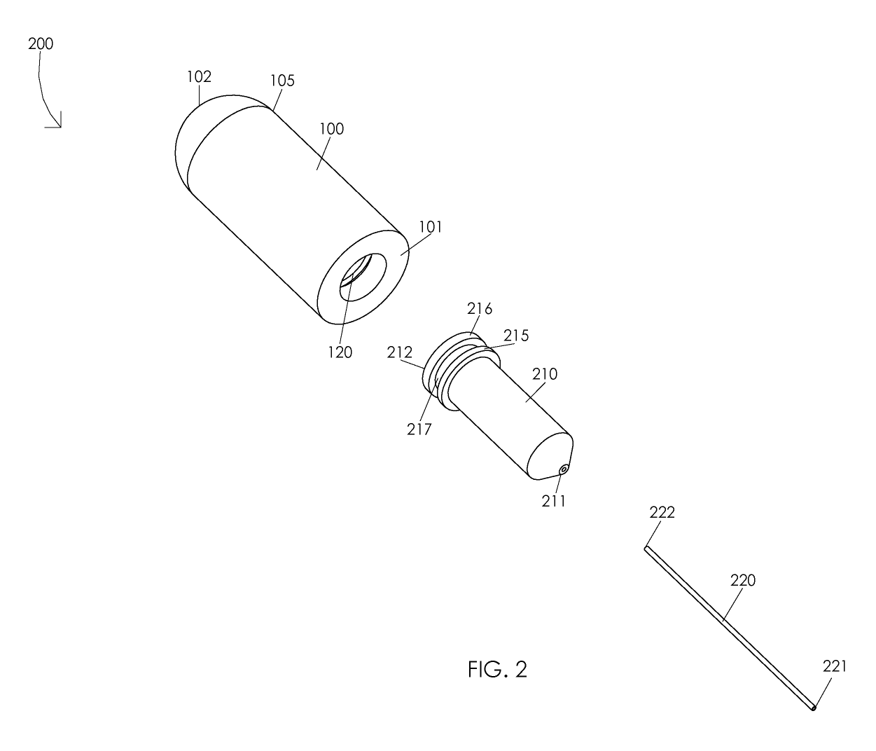

FIG. 2 is a schematic diagram illustrating an exploded view of a membrane visualization instrument assembly 200. Illustratively, a membrane visualization instrument assembly 200 may comprise a flow control mechanism 100, a visualization fluid guide 210 having a visualization fluid guide distal end 211 and a visualization fluid guide proximal end 212, and a hypodermic tube 220 having a hypodermic tube distal end 221 and a hypodermic tube proximal end 222. In one or more embodiments, hypodermic tube 220 may be manufactured with dimensions configured for performing microsurgical procedures, e.g., ophthalmic surgical procedures. Illustratively, hypodermic tube 220 may be manufactured with dimensions commonly used for ophthalmic surgical procedures, e.g., 20 gauge, 23 gauge, 25 gauge, 27 gauge, etc. In one or more embodiments, hypodermic tube 220 may have an outer diameter in a range of 0.01 to 0.032 inches. Illustratively, hypodermic tube 220 may have an outer diameter less than 0.01 inches or greater than 0.032 inches. In one or more embodiments, visualization fluid guide 210 may comprise a distal locking lip 215, a proximal locking lip 216, and a locking depression 217. Illustratively, visualization fluid guide 210 and hypodermic tube 220 may be manufactured from any suitable material, e.g., polymers, metals, metal alloys, etc., or from any combination of suitable materials.

In one or more embodiments, visualization fluid guide 210 may have a density in a range of 0.03 to 0.07 pounds per cubic inch, e.g., visualization fluid guide 210 may have a density of 0.051 pounds per cubic inch. Illustratively, visualization fluid guide 210 may have a density less than 0.03 pounds per cubic inch or greater than 0.07 pounds per cubic inch. In one or more embodiments, visualization fluid guide 210 may have a mass in a range of 0.0007 to 0.0021 pounds, e.g., visualization fluid guide 210 may have a mass of 0.0014 pounds. Illustratively, visualization fluid guide 210 may have a mass less than 0.0007 pounds or greater than 0.0021 pounds. In one or more embodiments, visualization fluid guide 210 may have a volume in a range of 0.01 to 0.04 cubic inches, e.g., visualization fluid guide 210 may have a volume of 0.0275 cubic inches. Illustratively, visualization fluid guide 210 may have a volume less than 0.01 cubic inches or greater than 0.04 cubic inches. In one or more embodiments, visualization fluid guide 210 may have a surface area in a range of 1.2 to 1.8 square inches, e.g., visualization fluid guide 210 may have a surface area of 1.57 square inches. Illustratively, visualization fluid guide 210 may have a surface area less than 1.2 square inches or greater than 1.8 square inches.

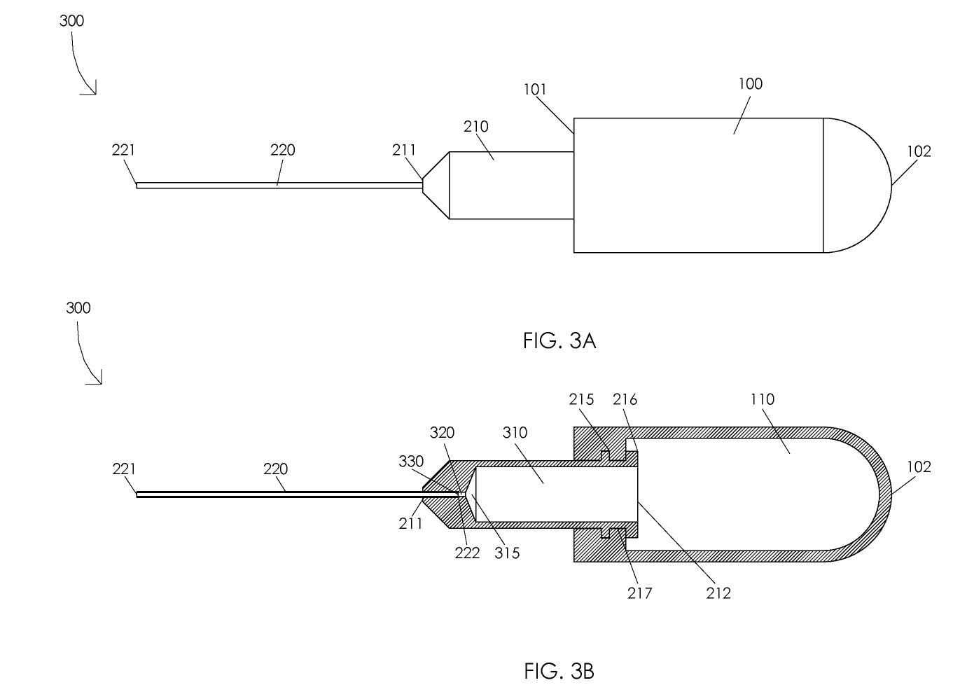

FIGS. 3A and 3B are schematic diagrams illustrating an assembled membrane visualization instrument 300. FIG. 3A illustrates a top view of an assembled membrane visualization instrument 300. FIG. 3B illustrates a cross-sectional view of an assembled membrane visualization instrument 300. Illustratively, an assembled membrane visualization instrument 300 may comprise a visualization fluid conduit 310, a visualization fluid conduit distal taper 315, a hermetic interface 320, and a hypodermic tube housing 330. Illustratively, a portion of visualization fluid guide 210 may be disposed within a portion of flow control mechanism 100, e.g., visualization fluid guide proximal end 212 may be disposed within visualization fluid chamber 110. In one or more embodiments, proximal locking lip 216 may be disposed within visualization fluid chamber 110, e.g., proximal locking lip 216 may be disposed between flow control mechanism proximal end 102 and flow control mechanism distal end 101. Illustratively, proximal locking lip 216 may be disposed within visualization fluid chamber 110, e.g., proximal locking lip 216 may be disposed between flow control mechanism proximal end 102 and visualization fluid guide housing 125. In one or more embodiments, proximal locking lip 216 may be disposed within visualization fluid chamber 110, e.g., proximal locking lip 216 may be disposed between flow control mechanism proximal end 102 and distal locking lip housing 120. Illustratively, proximal locking lip 216 may be disposed within visualization fluid chamber 110, e.g., proximal locking lip 216 may be disposed between flow control mechanism proximal end 102 and locking depression housing 115. In one or more embodiments, a portion of visualization fluid guide 210 may be disposed within a portion of flow control mechanism 100, e.g., a portion of visualization fluid guide 210 may be fixed within a portion of flow control mechanism 100. Illustratively, a portion of visualization fluid guide 210 may be fixed within a portion of flow control mechanism 100, e.g., proximal locking lip 216 may be fixed within visualization fluid chamber 110. In one or more embodiments, proximal locking lip 216 may be fixed within visualization fluid chamber 110, e.g., by an adhesive or any suitable fixation means. Illustratively, proximal locking lip 216 may be fixed within visualization fluid chamber 110, e.g., proximal locking lip 216 may be mechanically locked within visualization fluid chamber 110.

In one or more embodiments, locking depression 217 may be disposed within locking depression housing 115. Illustratively, locking depression 217 may be fixed within locking depression housing 115, e.g., by an adhesive or any suitable fixation means. Illustratively, locking depression 217 may be mechanically locked within locking depression housing 115. In one or more embodiments, distal locking lip 215 may be disposed within distal locking lip housing 120. Illustratively, distal locking lip 215 may be fixed within distal locking lip housing 120, e.g., by an adhesive or any suitable fixation means. In one or more embodiments, distal locking lip 215 may be mechanically fixed within distal locking lip housing 120. Illustratively, a portion of visualization fluid guide 210 may be disposed within a portion of flow control mechanism 100, e.g., a portion of visualization fluid guide 210 may be disposed within visualization fluid guide housing 125. In one or more embodiments, a portion of visualization fluid guide 210 may be fixed within visualization fluid guide housing 125, e.g., by an adhesive or any suitable fixation means. Illustratively, a portion of visualization fluid guide 210 may be mechanically fixed within visualization fluid guide housing 125.

In one or more embodiments, visualization fluid guide 210 may be disposed within flow control mechanism 100 wherein an interface between proximal locking lip 216 and an inner portion of visualization fluid chamber 110 comprises a hermetic seal. Illustratively, visualization fluid guide 210 may be disposed within flow control mechanism 100 wherein an interface between locking depression 217 and locking depression housing 115 comprises a hermetic seal. In one or more embodiments, visualization fluid guide 210 may be disposed within flow control mechanism 100 wherein an interface between distal locking lip 215 and distal locking lip housing 120 comprises a hermetic seal. Illustratively, visualization fluid guide 210 may be disposed within flow control mechanism 100 wherein an interface between a portion of visualization fluid guide 210 and visualization fluid guide housing 125 comprises a hermetic seal.

In one or more embodiments, a portion of hypodermic tube 220 may be disposed within a portion of visualization fluid guide 210, e.g., hypodermic tube proximal end 222 may be disposed within hypodermic tube housing 330. Illustratively, hypodermic tube 220 may be disposed within visualization fluid guide 210 wherein hypodermic tube proximal end 222 abuts hermetic interface 320. In one or more embodiments, hypodermic tube 220 may be disposed within visualization fluid guide 210 wherein an interface between hypodermic tube proximal end 222 and hermetic interface 320 comprises a hermetic seal. Illustratively, hypodermic tube 220 may be disposed within visualization fluid guide 210 wherein an interface between a portion of hypodermic tube 220 and hypodermic tube housing 330 comprises a hermetic seal. In one or more embodiments, a portion of hypodermic tube 220 may be fixed within a portion of visualization fluid guide 210, e.g., by an adhesive or any suitable fixation means. Illustratively, hypodermic tube 220 may be fixed within hypodermic tube housing 330 by a press fit.

In one or more embodiments, a surgeon may compress flow control mechanism 100, e.g., by applying a force to a portion of flow control mechanism 100. For example, a surgeon may compress flow control mechanism 100 by squeezing flow control mechanism 100. Illustratively, a compression of flow control mechanism 100 may be configured to reduce a volume of visualization fluid chamber 110. In one or more embodiments, a reduction of a volume of visualization fluid chamber 110 may be configured to increase a pressure within visualization fluid chamber 110. Illustratively, an increase of a pressure within visualization fluid chamber 110 may be configured to increase a pressure within visualization fluid conduit 310. In one or more embodiments, an increase of a pressure within visualization fluid conduit 310 may be configured to irrigate a gas or a fluid through hypodermic tube 220.

Illustratively, a surgeon may decompress flow control mechanism 100, e.g., by reducing a force applied to a portion of flow control mechanism 100. In one or more embodiments, a decompression of flow control mechanism 100 may be configured to increase a volume of visualization fluid chamber 110. Illustratively, an increase of a volume of visualization fluid chamber 110 may be configured to decrease a pressure within visualization fluid chamber 110. In one or more embodiments, a decrease of a pressure within visualization fluid chamber 110 may be configured to decrease a pressure within visualization fluid conduit 310. Illustratively, a decrease of a pressure within visualization fluid conduit 310 may be configured to aspirate a gas or a fluid through hypodermic tube 220.

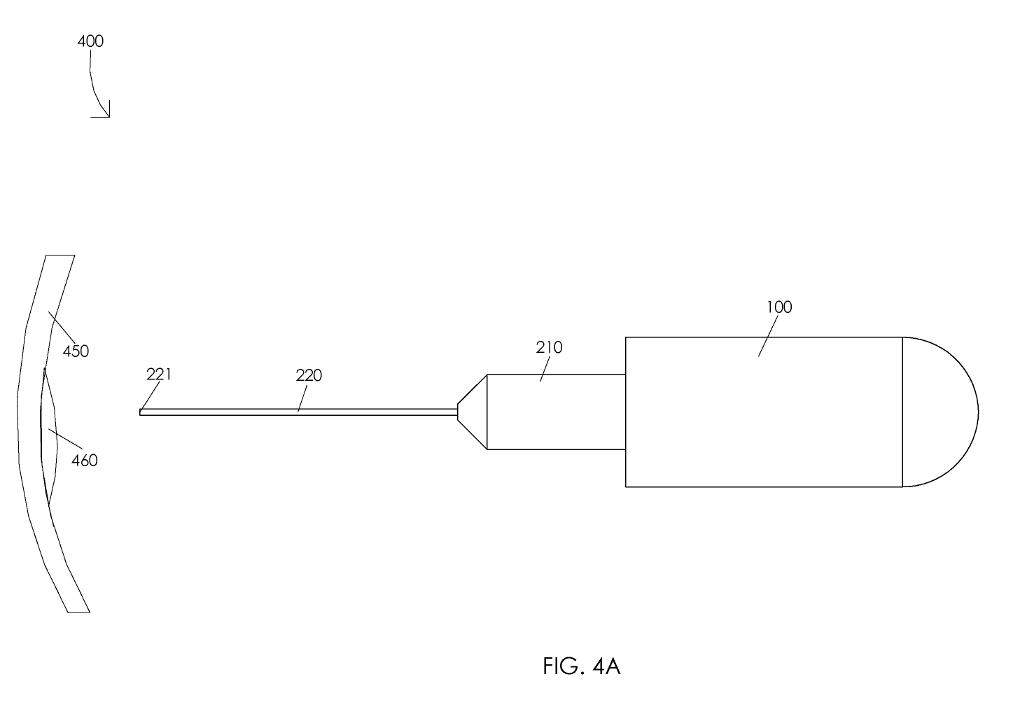

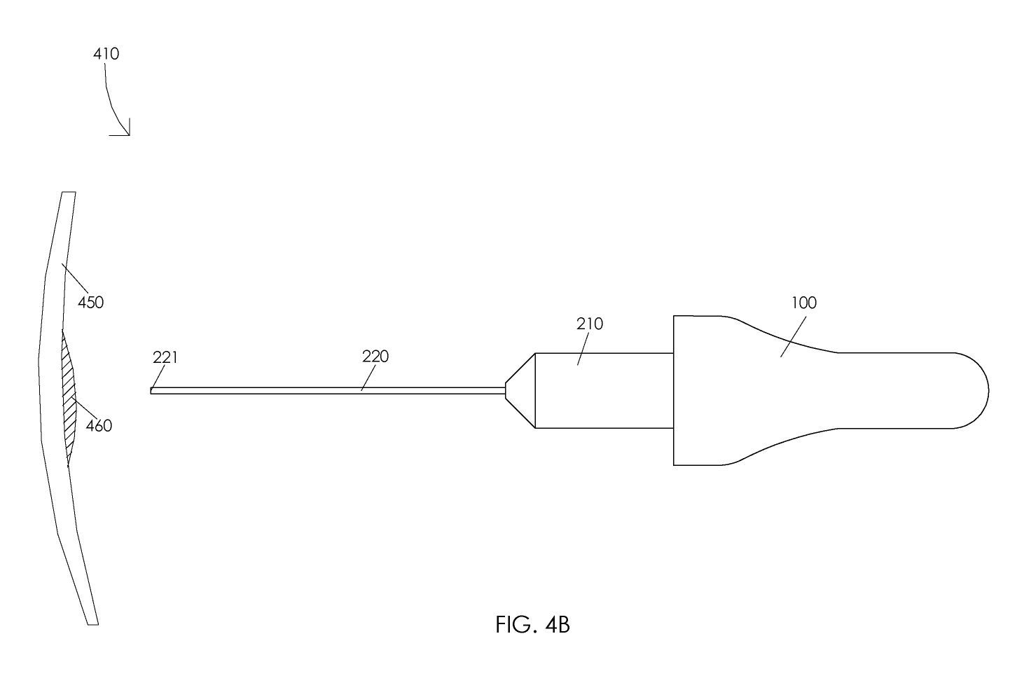

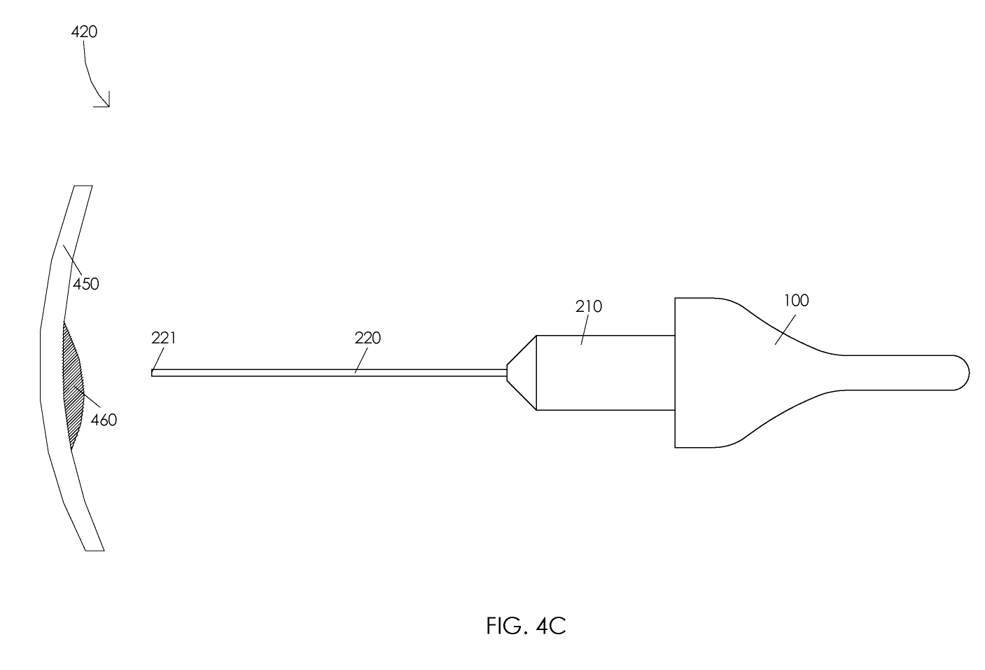

FIGS. 4A, 4B, and 4C are schematic diagrams illustrating an application of a visualization fluid to an internal limiting membrane 460. In one or more embodiments, internal limiting membrane 460 may be disposed over a retinal tissue 450. FIG. 4A illustrates a transparent membrane 400. Illustratively, internal limiting membrane 460 may comprise a transparent membrane 400, e.g., when flow control mechanism 100 is fully decompressed. In one or more embodiments, a visualization fluid, e.g., indocyanine green dye, kenalog, trypan blue dye, etc., may be disposed within visualization fluid chamber 110. Illustratively, a surgeon may insert hypodermic tube distal end 221 through an incision in an eye tissue, e.g., a surgeon may insert hypodermic tube 220 through a cannula in an eye tissue. In one or more embodiments, an intraocular pressure of an eye may be configured to prevent a visualization fluid from irrigating through hypodermic tube 220, e.g., when flow control mechanism 100 is fully decompressed.

FIG. 4B illustrates a partially stained membrane 410. Illustratively, a compression of flow control mechanism 100 may be configured to apply a visualization fluid to an internal limiting membrane 460. In one or more embodiments, an application of a visualization fluid to an internal limiting membrane 460 may be configured to allow a surgeon to visualize a transparent membrane 400 as a partially stained membrane 410. Illustratively, internal limiting membrane 460 may comprise a partially stained membrane 410, e.g., when a visualization fluid is partially applied to internal limiting membrane 460. In one or more embodiments, internal limiting membrane 460 may comprise a partially stained membrane 410, e.g., when flow control mechanism 100 is partially compressed. Illustratively, a compression of flow control mechanism 100 may be configured to decrease a volume of visualization fluid chamber 110. In one or more embodiments, a decrease of a volume of visualization fluid chamber 110 may increase a pressure within visualization fluid chamber 110. Illustratively, an increase of a pressure within visualization fluid chamber 110 may be configured to increase a pressure within visualization fluid conduit 310. In one or more embodiments, an increase of a pressure within visualization fluid chamber 110 and visualization fluid conduit 310 may be configured to irrigate a visualization fluid out of visualization fluid chamber 110 and into visualization fluid conduit 310. Illustratively, an increase in a pressure within visualization fluid chamber 110 and visualization fluid conduit 310 may be configured to irrigate a visualization fluid out of visualization fluid conduit 310 and into hypodermic tube 220. In one or more embodiments, visualization fluid conduit distal taper 315 may be configured to facilitate an irrigation of a visualization fluid out of irrigation fluid conduit 310 and into hypodermic tube 220. Illustratively, an increase in a pressure within visualization fluid chamber 110 and visualization fluid conduit 310 may be configured to irrigate a visualization fluid out of hypodermic tube distal end 221 and onto internal limiting membrane 460. In one or more embodiments, a compression of flow control mechanism 100 may be configured to irrigate a visualization fluid out of hypodermic tube distal end 221 and onto internal limiting membrane 460. Illustratively, a compression of flow control mechanism 100 may be configured to irrigate a visualization fluid out of hypodermic tube distal end 221 at a flow rate in a range of 0.1 to 10.0 milliliters per minute. In one or more embodiments, a compression of flow control mechanism 100 may be configured to irrigate a visualization fluid out of hypodermic tube distal end 221 at a flow rate less than 0.1 milliliters per minute or greater than 10.0 milliliters per minute. Illustratively, a compression of flow control mechanism 100 may be configured to irrigate a visualization fluid out of hypodermic tube distal end 221 in discrete volumes, e.g., a compression of flow control mechanism 100 may be configured to irrigate a visualization fluid out of hypodermic tube distal end 221 in discrete drops. In one or more embodiments, a compression of flow control mechanism 100 may be configured to irrigate a visualization fluid out of hypodermic tube distal end 221 in discrete drops wherein each discrete drop has a volume in a range of 0.001 to 0.08 milliliters, e.g., each discrete drop may have a volume of 0.05 milliliters. Illustratively, a compression of flow control mechanism 100 may be configured to irrigate a visualization fluid out of hypodermic tube distal end 221 in discrete drops wherein each drop has a volume less than 0.001 milliliters or greater than 0.08 milliliters. In one or more embodiments, an application of a first compressive force to a portion of flow control mechanism 100 may be configured to irrigate a first drop of a visualization fluid out of hypodermic tube distal end 221. Illustratively, an application of a second compressive force to a portion of flow control mechanism 100 may be configured to irrigate a second drop of a visualization fluid out of hypodermic tube distal end 221. In one or more embodiments, an application of a third compressive force to a portion of flow control mechanism 100 may be configured to irrigate a third drop of a visualization fluid out of hypodermic tube distal end 221. Illustratively, the second compressive force may have a greater magnitude than the first compressive force and the third compressive force may have a greater magnitude than the second compressive force. In one or more embodiments, a compression of flow control mechanism 100 may be configured to apply a visualization fluid to an internal limiting membrane 460, e.g., a compression of flow control mechanism 100 may be configured to allow a surgeon to visualize a transparent membrane 400 as a partially stained membrane 410.

FIG. 4C illustrates a fully stained membrane 420. Illustratively, a compression of flow control mechanism 100 may be configured to apply a visualization fluid to an internal limiting membrane 460. In one or more embodiments, an application of a visualization fluid to an internal limiting membrane 460 may be configured to allow a surgeon to visualize a partially stained membrane 410 as a fully stained membrane 420. Illustratively, internal limiting membrane 460 may comprise a fully stained membrane 420, e.g., when a visualization fluid is fully applied to internal limiting membrane 460. In one or more embodiments, internal limiting membrane 460 may comprise a fully stained membrane 420, e.g., when flow control mechanism 100 is fully compressed. Illustratively, a compression of flow control mechanism 100 may be configured to decrease a volume of visualization fluid chamber 110. In one or more embodiments, a decrease of a volume of visualization fluid chamber 110 may increase a pressure within visualization fluid chamber 110. Illustratively, an increase of a pressure within visualization fluid chamber 110 may be configured to increase a pressure within visualization fluid conduit 310. In one or more embodiments, an increase of a pressure within visualization fluid chamber 110 and visualization fluid conduit 310 may be configured to irrigate a visualization fluid out of visualization fluid chamber 110 and into visualization fluid conduit 310. Illustratively, an increase in a pressure within visualization fluid chamber 110 and visualization fluid conduit 310 may be configured to irrigate a visualization fluid out of visualization fluid conduit 310 and into hypodermic tube 220. In one or more embodiments, visualization fluid conduit distal taper 315 may be configured to facilitate an irrigation of a visualization fluid out of irrigation fluid conduit 310 and into hypodermic tube 220. Illustratively, an increase in a pressure within visualization fluid chamber 110 and visualization fluid conduit 310 may be configured to irrigate a visualization fluid out of hypodermic tube distal end 221 and onto internal limiting membrane 460. In one or more embodiments, a compression of flow control mechanism 100 may be configured to irrigate a visualization fluid out of hypodermic tube distal end 221 and onto internal limiting membrane 460. Illustratively, a compression of flow control mechanism 100 may be configured to irrigate a visualization fluid out of hypodermic tube distal end 221 at a flow rate in a range of 0.1 to 10.0 milliliters per minute. In one or more embodiments, a compression of flow control mechanism 100 may be configured to irrigate a visualization fluid out of hypodermic tube distal end 221 at a flow rate less than 0.1 milliliters per minute or greater than 10.0 milliliters per minute. Illustratively, a compression of flow control mechanism 100 may be configured to irrigate a visualization fluid out of hypodermic tube distal end 221 in discrete volumes, e.g., a compression of flow control mechanism 100 may be configured to irrigate a visualization fluid out of hypodermic tube distal end 221 in discrete drops. In one or more embodiments, a compression of flow control mechanism 100 may be configured to irrigate a visualization fluid out of hypodermic tube distal end 221 in discrete drops wherein each discrete drop has a volume in a range of 0.001 to 0.08 milliliters, e.g., each discrete drop may have a volume of 0.05 milliliters. Illustratively, a compression of flow control mechanism 100 may be configured to irrigate a visualization fluid out of hypodermic tube distal end 221 in discrete drops wherein each drop has a volume less than 0.001 milliliters or greater than 0.08 milliliters. In one or more embodiments, an application of a first compressive force to a portion of flow control mechanism 100 may be configured to irrigate a first drop of a visualization fluid out of hypodermic tube distal end 221. Illustratively, an application of a second compressive force to a portion of flow control mechanism 100 may be configured to irrigate a second drop of a visualization fluid out of hypodermic tube distal end 221. In one or more embodiments, an application of a third compressive force to a portion of flow control mechanism 100 may be configured to irrigate a third drop of a visualization fluid out of hypodermic tube distal end 221. Illustratively, the second compressive force may have a greater magnitude than the first compressive force and the third compressive force may have a greater magnitude than the second compressive force. In one or more embodiments, a compression of flow control mechanism 100 may be configured to apply a visualization fluid to an internal limiting membrane 460, e.g., a compression of flow control mechanism 100 may be configured to allow a surgeon to visualize a partially stained membrane 410 as a fully stained membrane 420.

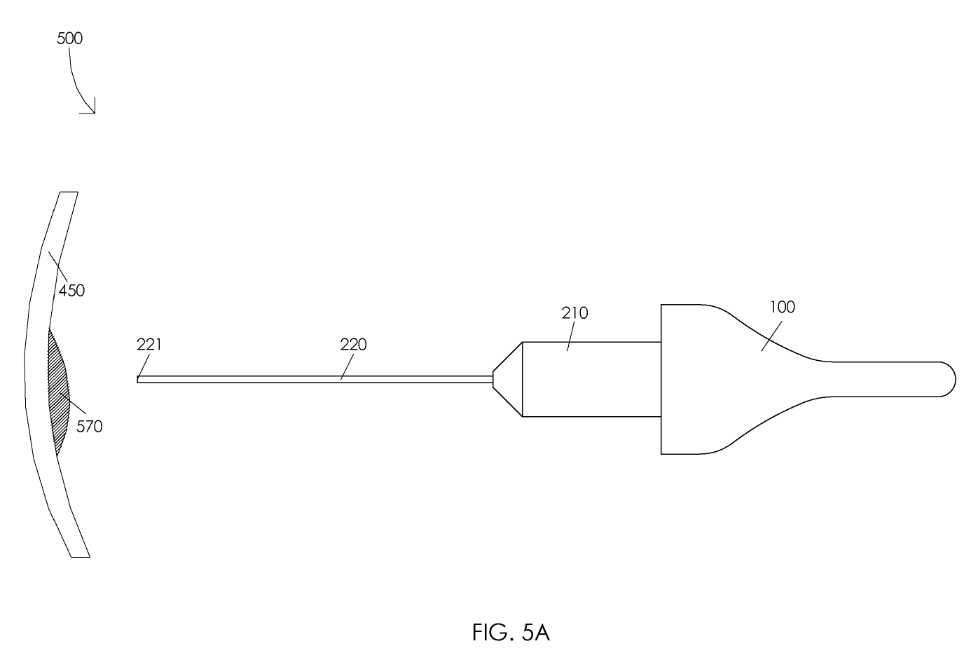

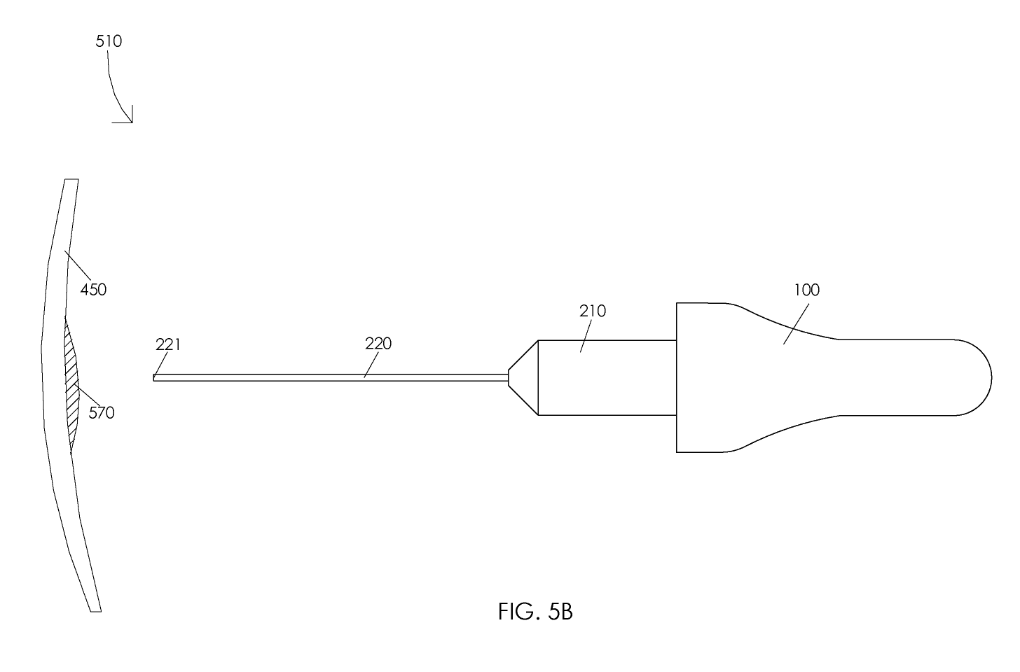

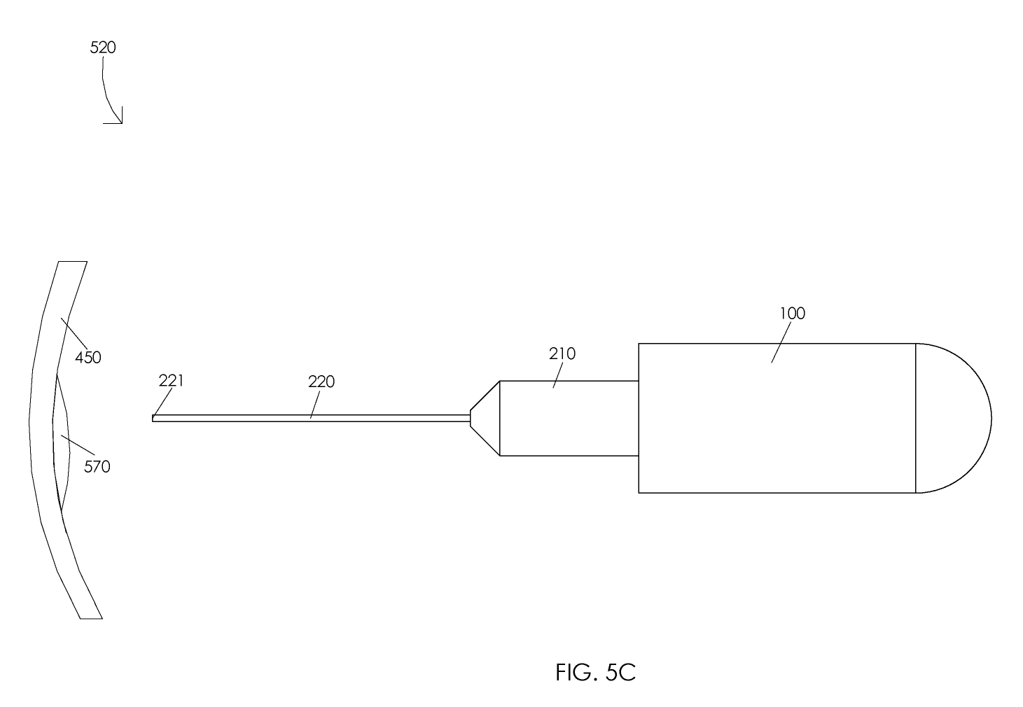

FIGS. 5A, 5B, and 5C are schematic diagrams illustrating a removal of a visualization fluid from a surgical site 570. FIG. 5A illustrates a surgical site with excess visualization fluid 500. In one or more embodiments, a visualization fluid, e.g., indocyanine green dye, kenalog, trypan blue dye, etc., may be at surgical site 570. Illustratively, surgical site 570 may comprise a surgical site with excess visualization fluid 500, e.g., when flow control mechanism 100 is fully compressed. In one or more embodiments, surgical site 570 may comprise a surgical site with excess visualization fluid 500, e.g., after an application of a visualization fluid to an internal limiting membrane 460. Illustratively, a surgical site with excess visualization fluid 500 may be adjacent to a retinal tissue 450, e.g., a surgical site with excess visualization fluid 500 may be adjacent to an internal limiting membrane 460.

FIG. 5B illustrates a surgical site with excess visualization fluid partially removed 510. Illustratively, a decompression of flow control mechanism 100 may be configured to remove a visualization fluid from a surgical site 570. In one or more embodiments, a removal of a visualization fluid from a surgical site 570 may be configured to allow a surgeon to visualize a surgical site with excess visualization fluid 500 as a surgical site with excess visualization fluid partially removed 510. Illustratively, surgical site 570 may comprise a surgical site with excess visualization fluid partially removed 510, e.g., when a visualization fluid is partially removed from surgical site 570. In one or more embodiments, surgical site 570 may comprise a surgical site with excess visualization fluid partially removed 510, e.g., when flow control mechanism 100 is partially decompressed. Illustratively, a decompression of flow control mechanism 100 may be configured to increase a volume of visualization fluid chamber 110. In one or more embodiments, an increase of a volume of visualization fluid chamber 110 may decrease a pressure within visualization fluid chamber 110. Illustratively, a decrease of a pressure within visualization fluid chamber 110 may be configured to decrease a pressure within visualization fluid conduit 310. In one or more embodiments, a decrease of a pressure within visualization fluid chamber 110 and visualization fluid conduit 310 may be configured to aspirate a visualization fluid out of surgical site 570 and into hypodermic tube 220. For example, a decrease of a pressure within visualization fluid chamber 110 and visualization fluid conduit 310 may be configured to aspirate a visualization fluid out of surgical site 570 and into hypodermic tube distal end 221. Illustratively, a decrease of a pressure within visualization fluid chamber 110 and visualization fluid conduit 310 may be configured to aspirate a visualization fluid through hypodermic tube 220 and out of hypodermic tube proximal end 222. In one or more embodiments, a decrease of a pressure within visualization fluid chamber 110 and visualization fluid conduit 310 may be configured to aspirate a visualization fluid out of hypodermic tube proximal end 222 and into visualization fluid conduit 310. Illustratively, a decrease of a pressure within visualization fluid chamber 110 and visualization fluid conduit 310 may be configured to aspirate a visualization fluid out of visualization fluid conduit 310 and into visualization fluid chamber 110. In one or more embodiments, a decompression of flow control mechanism 100 may be configured to aspirate a visualization fluid into hypodermic tube distal end 221 at a flow rate in a range of 0.06 to 41.0 milliliters per minute. Illustratively, a decompression of flow control mechanism 100 may be configured to aspirate a visualization fluid into hypodermic tube distal end 221 at a flow rate less than 0.06 milliliters per minute or greater than 41.0 milliliters per minute. In one or more embodiments, a decompression of flow control mechanism 100 may be configured to remove a visualization fluid from a surgical site 570, e.g., a decompression of flow control mechanism 100 may be configured to allow a surgeon to visualize a surgical site with excess visualization fluid 500 as a surgical site with excess visualization fluid partially removed 510.

FIG. 5C illustrates a surgical site with excess visualization fluid completely removed 520. Illustratively, a decompression of flow control mechanism 100 may be configured to remove a visualization fluid from a surgical site 570. In one or more embodiments, a removal of a visualization fluid from a surgical site 570 may be configured to allow a surgeon to visualize a surgical site with excess visualization fluid partially removed 510 as a surgical site with excess visualization fluid completely removed 520. Illustratively, surgical site 570 may comprise a surgical site with excess visualization fluid completely removed 520, e.g., when a visualization fluid is completely removed from surgical site 570. In one or more embodiments, surgical site 570 may comprise a surgical site with excess visualization fluid completely removed 520, e.g., when flow control mechanism 100 is fully decompressed. Illustratively, a decompression of flow control mechanism 100 may be configured to increase a volume of visualization fluid chamber 110. In one or more embodiments, an increase of a volume of visualization fluid chamber 110 may decrease a pressure within visualization fluid chamber 110. Illustratively, a decrease of a pressure within visualization fluid chamber 110 may be configured to decrease a pressure within visualization fluid conduit 310. In one or more embodiments, a decrease of a pressure within visualization fluid chamber 110 and visualization fluid conduit 310 may be configured to aspirate a visualization fluid out of surgical site 570 and into hypodermic tube 220. For example, a decrease of a pressure within visualization fluid chamber 110 and visualization fluid conduit 310 may be configured to aspirate a visualization fluid out of surgical site 570 and into hypodermic tube distal end 221. Illustratively, a decrease of a pressure within visualization fluid chamber 110 and visualization fluid conduit 310 may be configured to aspirate a visualization fluid through hypodermic tube 220 and out of hypodermic tube proximal end 222. In one or more embodiments, a decrease of a pressure within visualization fluid chamber 110 and visualization fluid conduit 310 may be configured to aspirate a visualization fluid out of hypodermic tube proximal end 222 and into visualization fluid conduit 310. Illustratively, a decrease of a pressure within visualization fluid chamber 110 and visualization fluid conduit 310 may be configured to aspirate a visualization fluid out of visualization fluid conduit 310 and into visualization fluid chamber 110. In one or more embodiments, a decompression of flow control mechanism 100 may be configured to aspirate a visualization fluid into hypodermic tube distal end 221 at a flow rate in a range of 0.06 to 41.0 milliliters per minute. Illustratively, a decompression of flow control mechanism 100 may be configured to aspirate a visualization fluid into hypodermic tube distal end 221 at a flow rate less than 0.06 milliliters per minute or greater than 41.0 milliliters per minute. In one or more embodiments, a decompression of flow control mechanism 100 may be configured to remove a visualization fluid from a surgical site 570, e.g., a decompression of flow control mechanism 100 may be configured to allow a surgeon to visualize a surgical site with excess visualization fluid partially removed 510 as a surgical site with excess visualization fluid completely removed 520.

Illustratively, one or more properties of a membrane visualization instrument may be adjusted to attain one or more desired membrane visualization instrument features. In one or more embodiments, membrane visualization instrument may be configured to apply a visualization fluid to an epiretinal membrane, e.g., a surgeon may compress flow control mechanism 100 to irrigate a visualization fluid out of hypodermic tube distal end 221 and onto an epiretinal membrane. Illustratively, an epiretinal membrane may comprise a transparent membrane 400, e.g., when flow control mechanism 100 is fully decompressed. In one or more embodiments, a compression of flow control mechanism 100 may be configured to irrigate a visualization fluid out of hypodermic tube distal end 221 and onto an epiretinal membrane. Illustratively, an application of a visualization fluid to an epiretinal membrane may be configured to allow a surgeon to visualize a transparent membrane 400 as a partially stained membrane 410. In one or more embodiments, an epiretinal membrane may comprise a partially stained membrane 410, e.g., when a visualization fluid is partially applied to the epiretinal membrane. Illustratively, compression of flow control mechanism 100 may be configured to irrigate a visualization fluid out of hypodermic tube distal end 221 and onto an epiretinal membrane. In one or more embodiments, an application of a visualization fluid to an epiretinal membrane may be configured to allow a surgeon to visualize a partially stained membrane 410 as a fully stained membrane 420. Illustratively, an epiretinal membrane may comprise a fully stained membrane 420, e.g., when a visualization fluid is fully applied to the epiretinal membrane.

In one or more embodiments, a volume of visualization fluid chamber 110 may be adjusted, e.g., to vary an amount of a visualization fluid available for applying to internal limiting membrane 460 or an epiretinal membrane. Illustratively, a stiffness of flow control mechanism 100 may be adjusted, e.g., to vary a magnitude of a compressive force configured to irrigate a particular volume of a visualization fluid out of hypodermic tube distal end 221. In one or more embodiments, flow control mechanism 100 may comprise a transparent material configured to allow a surgeon to visualize an amount of a visualization fluid disposed within visualization fluid chamber 110, e.g., flow control mechanism 100 may comprise a transparent silicon material. Illustratively, an inner diameter of hypodermic tube 220 may be adjusted, e.g., to vary a visualization fluid irrigation flow rate. In one or more embodiments, an inner diameter of hypodermic tube 220 may be adjusted, e.g., to vary a visualization fluid aspiration flow rate. Illustratively, hypodermic tube 220 may have an inner diameter in a range of 0.009 to 0.031 inches. In one or more embodiments, hypodermic tube 220 may have an inner diameter less than 0.009 inches or greater than 0.031 inches.

Illustratively, a visualization fluid, e.g., indocyanine green dye, kenalog, trypan blue dye, etc., may be disposed within visualization fluid chamber 110. In one or more embodiments, a visualization fluid may be disposed within a hermetically sealed capsule, e.g., a visualization fluid may be disposed within a hermetically sealed pouch. Illustratively, the hermetically sealed capsule may be disposed within visualization fluid chamber 110, e.g., the hermetically sealed capsule may be disposed between visualization fluid guide proximal end 212 and flow control mechanism proximal end 102. In one or more embodiments, a portion of the hermetically sealed capsule may be disposed within visualization fluid conduit 310, e.g., the hermetically sealed capsule may be disposed between visualization fluid guide distal end 211 and flow control mechanism proximal end 102. Illustratively, a compression of flow control mechanism 100 may be configured to rupture the hermetically sealed capsule and release a visualization fluid within visualization fluid chamber 110. In one or more embodiments, a first compression of flow control mechanism 100 may be configured to release a visualization fluid within visualization fluid chamber 110 and a second compression of flow control mechanism 100 may be configured to irrigate the visualization fluid through hypodermic tube 220 and out of hypodermic tube distal end 221.

Illustratively, a visualization fluid may comprise two or more component fluids, e.g., two or more component fluids may be combined. In one or more embodiments, a first component fluid may be disposed within a first hermetically sealed capsule and a second component fluid may be disposed within a second hermetically sealed capsule. Illustratively, the first hermetically sealed capsule and the second hermetically sealed capsule may be disposed within visualization fluid chamber 110. In one or more embodiments, a compression of flow control mechanism 100 may be configured to rupture the first hermetically sealed capsule and the second hermetically sealed capsule. Illustratively, a rupture of the first hermetically sealed capsule and the second hermetically sealed capsule may release the first component fluid and the second component fluid within visualization fluid chamber 110 wherein the first component fluid and the second component fluid are combined. In one or more embodiments, a first compression of flow control mechanism 100 may be configured to release a first fluid component and a second fluid component within visualization fluid chamber 110 and a second compression of flow control mechanism 100 may be configured to irrigate a visualization fluid through hypodermic tube 220 and out of hypodermic tube distal end 221.

Illustratively, a visualization fluid, e.g., indocyanine green dye, kenalog, trypan blue dye, etc., may be external to a membrane visualization instrument, e.g., a visualization fluid may be disposed within a container in an operating room. In one or more embodiments, a surgeon or a surgeon's assistant may decompress flow control mechanism 100, e.g., to aspirate a visualization fluid out of a container and into hypodermic tube distal end 221. Illustratively, a surgeon or a surgeon's assistant may decompress flow control mechanism 100, e.g., to aspirate a visualization fluid out of hypodermic tube proximal end 222 and into visualization fluid conduit 310. In one or more embodiments, a surgeon or a surgeon's assistant may decompress flow control mechanism 100, e.g., to aspirate a visualization fluid out of visualization fluid conduit 310 and into visualization fluid chamber 110. Illustratively, a surgeon may compress flow control mechanism 100, e.g., to apply a visualization fluid to a transparent membrane 400.

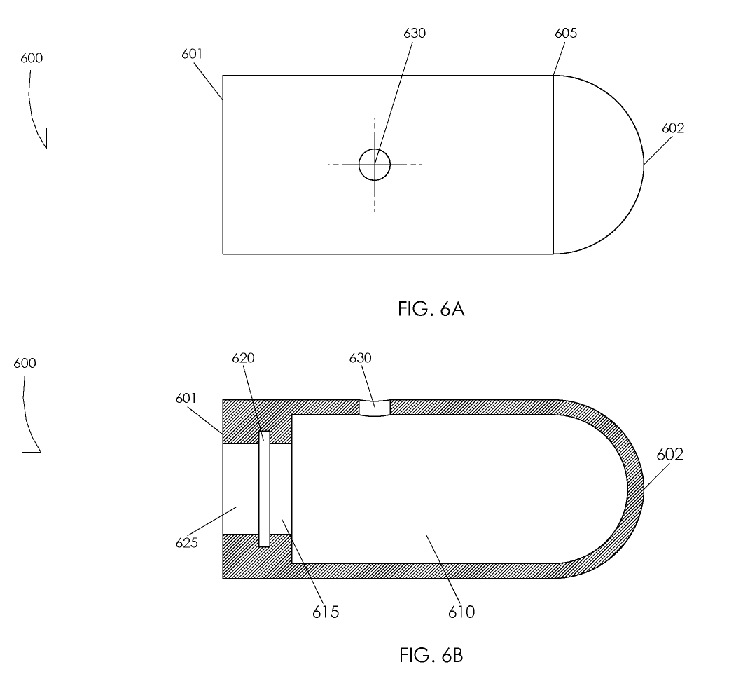

FIGS. 6A and 6B are schematic diagrams illustrating a flow control mechanism 600. FIG. 6A illustrates a top view of a flow control mechanism 600. Illustratively, flow control mechanism 600 may comprise a flow control mechanism distal end 601, a flow control mechanism proximal end 602, and a flow control mechanism dome interface 605, and a pressure vent 630. In one or more embodiments, flow control mechanism 600 may be manufactured from any suitable material, e.g., polymers, metals, metal alloys, etc., or from any combination of suitable materials.

FIG. 6B illustrates a cross-sectional view of a flow control mechanism 600. Illustratively, flow control mechanism 600 may comprise a visualization fluid chamber 610, a locking depression housing 615, a distal locking lip housing 620, and a visualization fluid guide housing 625. In one or more embodiments, flow control mechanism 600 may have a density in a range of 0.02 to 0.06 pounds per cubic inch, e.g., flow control mechanism 600 may have a density of 0.0399 pounds per cubic inch. Illustratively, flow control mechanism 600 may have a density less than 0.02 pounds per cubic inch or greater than 0.06 pounds per cubic inch. In one or more embodiments, flow control mechanism 600 may have a mass in a range of 0.003 to 0.007 pounds, e.g., flow control mechanism 600 may have a mass of 0.005 pounds. Illustratively, flow control mechanism 600 may have a mass less than 0.003 pounds or greater than 0.007 pounds. In one or more embodiments, flow control mechanism 600 may have a volume in a range of 0.06 to 0.18 cubic inches, e.g., flow control mechanism 600 may have a volume of 0.126 cubic inches. Illustratively, flow control mechanism 600 may have a volume less than 0.06 cubic inches or greater than 0.18 cubic inches. In one or more embodiments, flow control mechanism 600 may have a surface area in a range of 4.4 to 4.8 square inches, e.g., flow control mechanism 600 may have a surface area of 4.6 square inches. Illustratively, flow control mechanism 600 may have a surface area less than 4.4 square inches or greater than 4.8 square inches.

In one or more embodiments, flow control mechanism 600 may be manufactured from a material suitable for sterilization by a medical autoclave. Illustratively, flow control mechanism 600 may be manufactured from a material, e.g., rubber, configured to withstand exposure to temperatures, pressures, and ambient conditions present in a medical autoclave without degradation. For example, flow control mechanism 600 may be configured to function normally after exposure in a temperature 250° F. for 15 minutes at an atmospheric pressure of 15 psi. In one or more embodiments, flow control mechanism 600 may be configured to be used in a surgical procedure and then sterilized by a medical autoclave at least three times. Illustratively, flow control mechanism 600 may be configured to be used in a surgical procedure and then sterilized by a medical autoclave more than three times.

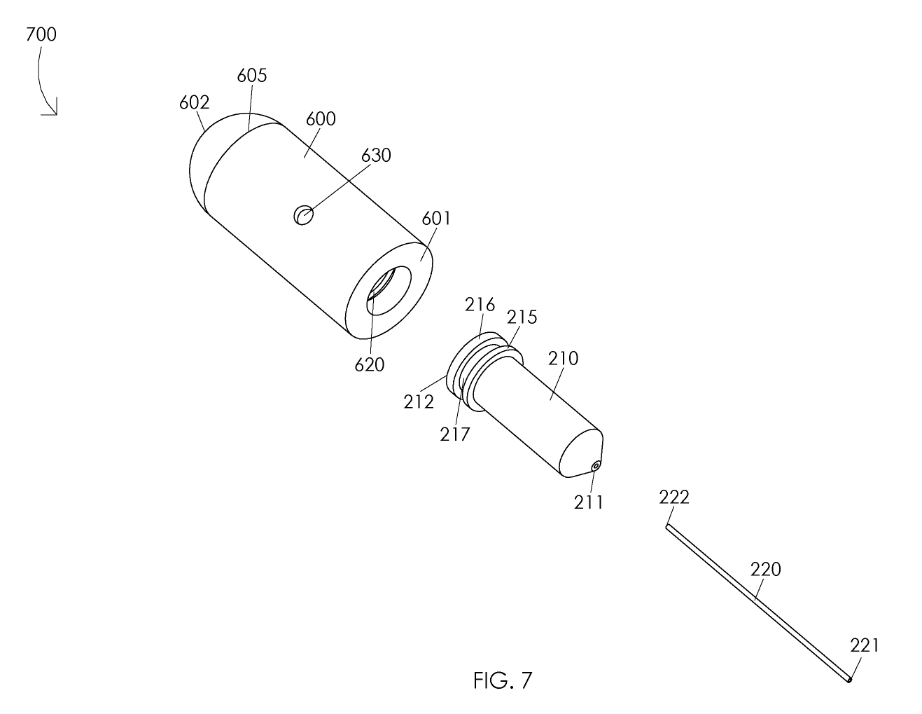

FIG. 7 is a schematic diagram illustrating an exploded view of a membrane visualization instrument assembly 700. Illustratively, a membrane visualization instrument assembly 700 may comprise a flow control mechanism 600, a visualization fluid guide 210 having a visualization fluid guide distal end 211 and a visualization fluid guide proximal end 212, and a hypodermic tube 220 having a hypodermic tube distal end 221 and a hypodermic tube proximal end 222. In one or more embodiments, hypodermic tube 220 may be manufactured with dimensions configured for performing microsurgical procedures, e.g., ophthalmic surgical procedures. Illustratively, hypodermic tube 220 may be manufactured with dimensions commonly used for ophthalmic surgical procedures, e.g., 20 gauge, 23 gauge, 25 gauge, 27 gauge, etc. In one or more embodiments, hypodermic tube 220 may have an outer diameter in a range of 0.01 to 0.032 inches. Illustratively, hypodermic tube 220 may have an outer diameter less than 0.01 inches or greater than 0.032 inches. In one or more embodiments, visualization fluid guide 210 may comprise a distal locking lip 215, a proximal locking lip 216, and a locking depression 217. Illustratively, visualization fluid guide 210 and hypodermic tube 220 may be manufactured from any suitable material, e.g., polymers, metals, metal alloys, etc., or from any combination of suitable materials.

In one or more embodiments, visualization fluid guide 210 may have a density in a range of 0.03 to 0.07 pounds per cubic inch, e.g., visualization fluid guide 210 may have a density of 0.051 pounds per cubic inch. Illustratively, visualization fluid guide 210 may have a density less than 0.03 pounds per cubic inch or greater than 0.07 pounds per cubic inch. In one or more embodiments, visualization fluid guide 210 may have a mass in a range of 0.0007 to 0.0021 pounds, e.g., visualization fluid guide 210 may have a mass of 0.0014 pounds. Illustratively, visualization fluid guide 210 may have a mass less than 0.0007 pounds or greater than 0.0021 pounds. In one or more embodiments, visualization fluid guide 210 may have a volume in a range of 0.01 to 0.04 cubic inches, e.g., visualization fluid guide 210 may have a volume of 0.0275 cubic inches. Illustratively, visualization fluid guide 210 may have a volume less than 0.01 cubic inches or greater than 0.04 cubic inches. In one or more embodiments, visualization fluid guide 210 may have a surface area in a range of 1.2 to 1.8 square inches, e.g., visualization fluid guide 210 may have a surface area of 1.57 square inches. Illustratively, visualization fluid guide 210 may have a surface area less than 1.2 square inches or greater than 1.8 square inches.

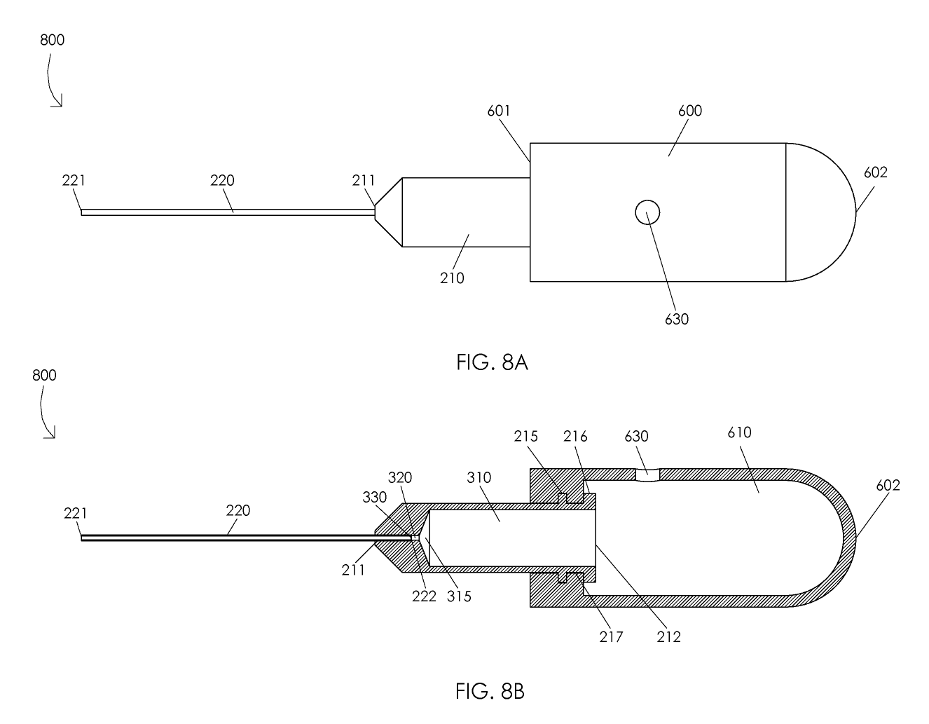

FIGS. 8A and 8B are schematic diagrams illustrating an assembled membrane visualization instrument 800. FIG. 8A illustrates a top view of an assembled membrane visualization instrument 800. FIG. 8B illustrates a cross-sectional view of an assembled membrane visualization instrument 800. Illustratively, an assembled membrane visualization instrument 800 may comprise a visualization fluid conduit 310, a visualization fluid conduit distal taper 315, a hermetic interface 320, and a hypodermic tube housing 330. Illustratively, a portion of visualization fluid guide 210 may be disposed within a portion of flow control mechanism 600, e.g., visualization fluid guide proximal end 212 may be disposed within visualization fluid chamber 610. In one or more embodiments, proximal locking lip 216 may be disposed within visualization fluid chamber 610, e.g., proximal locking lip 216 may be disposed between flow control mechanism proximal end 602 and flow control mechanism distal end 601. Illustratively, proximal locking lip 216 may be disposed within visualization fluid chamber 610, e.g., proximal locking lip 216 may be disposed between flow control mechanism proximal end 602 and visualization fluid guide housing 625. In one or more embodiments, proximal locking lip 216 may be disposed within visualization fluid chamber 610, e.g., proximal locking lip 216 may be disposed between flow control mechanism proximal end 602 and distal locking lip housing 620. Illustratively, proximal locking lip 216 may be disposed within visualization fluid chamber 610, e.g., proximal locking lip 216 may be disposed between flow control mechanism proximal end 602 and locking depression housing 615. In one or more embodiments, a portion of visualization fluid guide 210 may be disposed within a portion of flow control mechanism 600, e.g., a portion of visualization fluid guide 210 may be fixed within a portion of flow control mechanism 600. Illustratively, a portion of visualization fluid guide 210 may be fixed within a portion of flow control mechanism 600, e.g., proximal locking lip 216 may be fixed within visualization fluid chamber 610. In one or more embodiments, proximal locking lip 216 may be fixed within visualization fluid chamber 610, e.g., by an adhesive or any suitable fixation means. Illustratively, proximal locking lip 216 may be fixed within visualization fluid chamber 610, e.g., proximal locking lip 216 may be mechanically locked within visualization fluid chamber 610.

In one or more embodiments, locking depression 217 may be disposed within locking depression housing 615. Illustratively, locking depression 217 may be fixed within locking depression housing 615, e.g., by an adhesive or any suitable fixation means. Illustratively, locking depression 217 may be mechanically locked within locking depression housing 615. In one or more embodiments, distal locking lip 215 may be disposed within distal locking lip housing 620. Illustratively, distal locking lip 215 may be fixed within distal locking lip housing 620, e.g., by an adhesive or any suitable fixation means. In one or more embodiments, distal locking lip 215 may be mechanically fixed within distal locking lip housing 620. Illustratively, a portion of visualization fluid guide 210 may be disposed within a portion of flow control mechanism 600, e.g., a portion of visualization fluid guide 210 may be disposed within visualization fluid guide housing 625. In one or more embodiments, a portion of visualization fluid guide 210 may be fixed within visualization fluid guide housing 625, e.g., by an adhesive or any suitable fixation means. Illustratively, a portion of visualization fluid guide 210 may be mechanically fixed within visualization fluid guide housing 625.

In one or more embodiments, visualization fluid guide 210 may be disposed within flow control mechanism 600 wherein an interface between proximal locking lip 216 and an inner portion of visualization fluid chamber 610 comprises a hermetic seal. Illustratively, visualization fluid guide 210 may be disposed within flow control mechanism 600 wherein an interface between locking depression 217 and locking depression housing 615 comprises a hermetic seal. In one or more embodiments, visualization fluid guide 210 may be disposed within flow control mechanism 600 wherein an interface between distal locking lip 215 and distal locking lip housing 620 comprises a hermetic seal. Illustratively, visualization fluid guide 210 may be disposed within flow control mechanism 600 wherein an interface between a portion of visualization fluid guide 210 and visualization fluid guide housing 625 comprises a hermetic seal.

In one or more embodiments, a portion of hypodermic tube 220 may be disposed within a portion of visualization fluid guide 210, e.g., hypodermic tube proximal end 222 may be disposed within hypodermic tube housing 330. Illustratively, hypodermic tube 220 may be disposed within visualization fluid guide 210 wherein hypodermic tube proximal end 222 abuts hermetic interface 320. In one or more embodiments, hypodermic tube 220 may be disposed within visualization fluid guide 210 wherein an interface between hypodermic tube proximal end 222 and hermetic interface 320 comprises a hermetic seal. Illustratively, hypodermic tube 220 may be disposed within visualization fluid guide 210 wherein an interface between a portion of hypodermic tube 220 and hypodermic tube housing 330 comprises a hermetic seal. In one or more embodiments, a portion of hypodermic tube 220 may be fixed within a portion of visualization fluid guide 210, e.g., by an adhesive or any suitable fixation means. Illustratively, hypodermic tube 220 may be fixed within hypodermic tube housing 330 by a press fit.

In one or more embodiments, a surgeon may compress flow control mechanism 600, e.g., by applying a force to a portion of flow control mechanism 600. For example, a surgeon may compress flow control mechanism 600 by squeezing flow control mechanism 600. Illustratively, a compression of flow control mechanism 600 may be configured to reduce a volume of visualization fluid chamber 610. In one or more embodiments, a reduction of a volume of visualization fluid chamber 610 may be configured to increase a pressure within visualization fluid chamber 610. Illustratively, an increase of a pressure within visualization fluid chamber 610 may be configured to increase a pressure within visualization fluid conduit 310. In one or more embodiments, an increase of a pressure within visualization fluid conduit 310 may be configured to irrigate a gas or a fluid through hypodermic tube 220.

In one or more embodiments, a surgeon my cover pressure vent 630, e.g., a surgeon may cover pressure vent 630 with a portion of the surgeon's hand. Illustratively, a surgeon may cover pressure vent 630, e.g., a surgeon may cover pressure vent 630 with a portion of a thumb or a finger. In one or more embodiments, a surgeon may cover pressure vent 630 wherein a covered pressure vent 630 comprises a hermetic seal. In one or more embodiments, a surgeon may selectively control an irrigation flow rate of a gas or fluid, e.g., by covering or exposing a portion of pressure vent 630. Illustratively, a surgeon may selectively decrease a pressure within visualization fluid chamber 610, e.g., by exposing a portion of pressure vent 630. In one or more embodiments, a surgeon may selectively decrease an irrigation flow rate of a gas or fluid, e.g., by exposing a portion of pressure vent 630. Illustratively, a surgeon may selectively increase a pressure within visualization fluid chamber 610, e.g., by covering a portion of pressure vent 630. In one or more embodiments, a surgeon may selectively increase an irrigation flow rate of a gas or fluid, e.g., by covering a portion of pressure vent 630.

Illustratively, a surgeon may decompress flow control mechanism 600, e.g., by reducing a force applied to a portion of flow control mechanism 600. In one or more embodiments, a decompression of flow control mechanism 600 may be configured to increase a volume of visualization fluid chamber 610. Illustratively, an increase of a volume of visualization fluid chamber 610 may be configured to decrease a pressure within visualization fluid chamber 610. In one or more embodiments, a decrease of a pressure within visualization fluid chamber 610 may be configured to decrease a pressure within visualization fluid conduit 310. Illustratively, a decrease of a pressure within visualization fluid conduit 310 may be configured to aspirate a gas or a fluid through hypodermic tube 220.

In one or more embodiments, a surgeon my cover pressure vent 630, e.g., a surgeon may cover pressure vent 630 with a portion of the surgeon's hand. Illustratively, a surgeon may cover pressure vent 630, e.g., a surgeon may cover pressure vent 630 with a portion of a thumb or a finger. In one or more embodiments, a surgeon may cover pressure vent 630 wherein a covered pressure vent 630 comprises a hermetic seal. In one or more embodiments, a surgeon may selectively control an aspiration flow rate of a gas or fluid, e.g., by covering or exposing a portion of pressure vent 630. Illustratively, a surgeon may selectively decrease a pressure within visualization fluid chamber 610, e.g., by exposing a portion of pressure vent 630. In one or more embodiments, a surgeon may selectively increase an aspiration flow rate of a gas or fluid, e.g., by exposing a portion of pressure vent 630. Illustratively, a surgeon may selectively increase a pressure within visualization fluid chamber 610, e.g., by covering a portion of pressure vent 630. In one or more embodiments, a surgeon may selectively decrease an aspiration flow rate of a gas or fluid, e.g., by covering a portion of pressure vent 630.

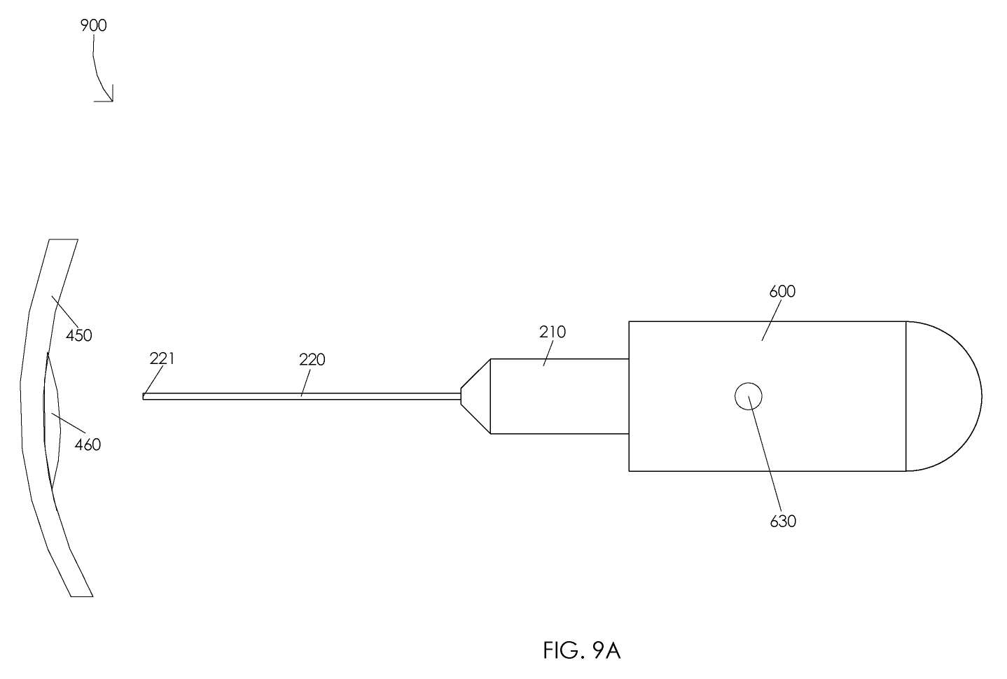

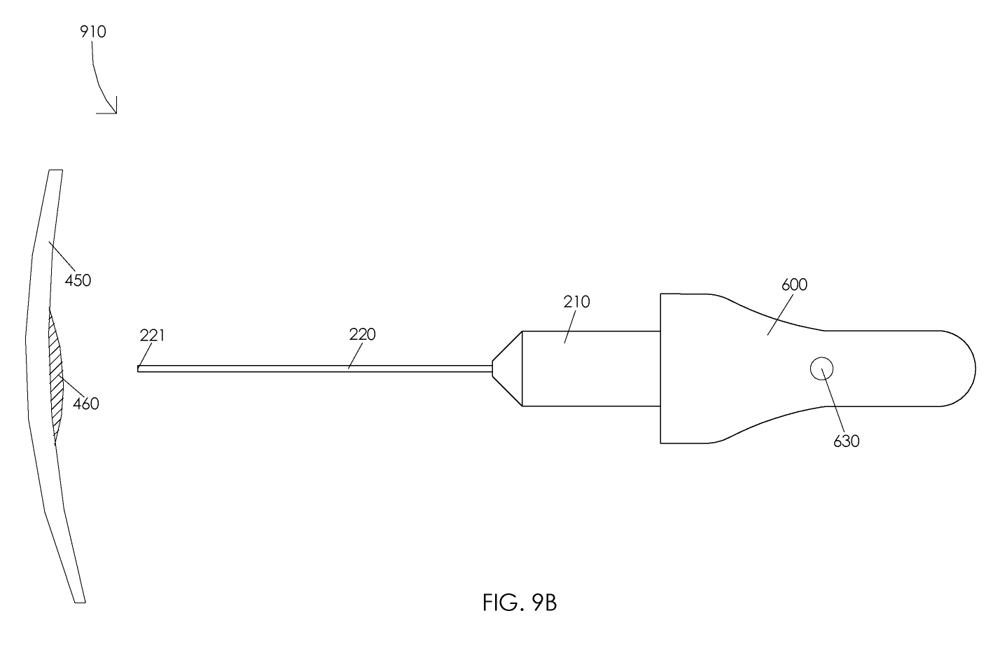

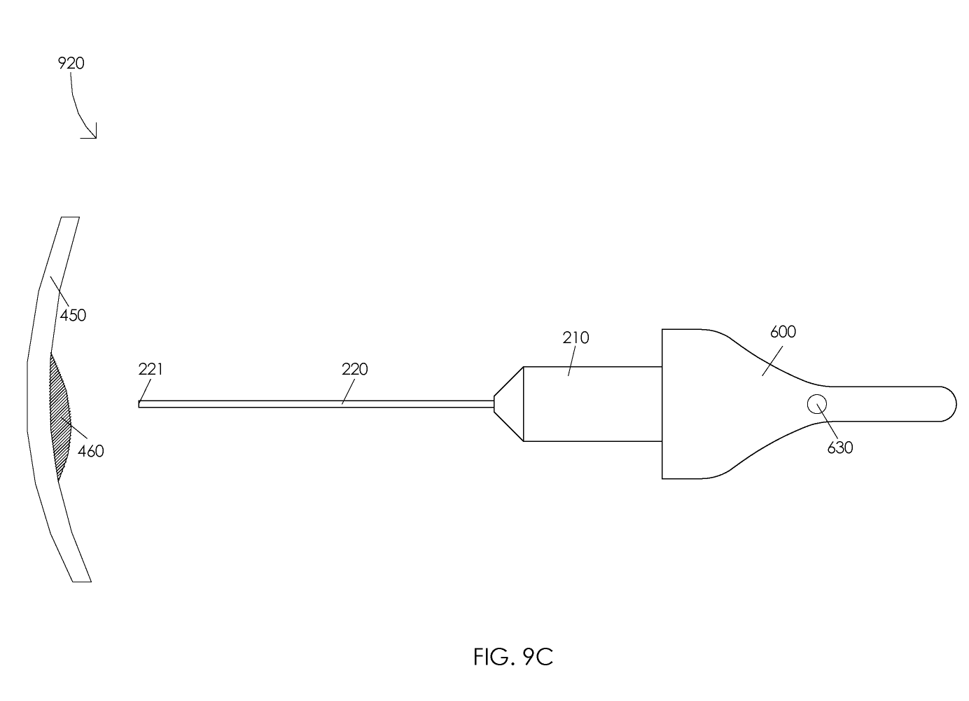

FIGS. 9A, 9B, and 9C are schematic diagrams illustrating an application of a visualization fluid to an internal limiting membrane 460. In one or more embodiments, internal limiting membrane 460 may be disposed over a retinal tissue 450. FIG. 9A illustrates a transparent membrane 900. Illustratively, internal limiting membrane 460 may comprise a transparent membrane 900, e.g., when flow control mechanism 600 is fully decompressed. In one or more embodiments, a visualization fluid, e.g., indocyanine green dye, kenalog, trypan blue dye, etc., may be disposed within visualization fluid chamber 610. Illustratively, a surgeon may insert hypodermic tube distal end 221 through an incision in an eye tissue, e.g., a surgeon may insert hypodermic tube 220 through a cannula in an eye tissue. In one or more embodiments, an intraocular pressure of an eye may be configured to prevent a visualization fluid from irrigating through hypodermic tube 220, e.g., when flow control mechanism 600 is fully decompressed.

FIG. 9B illustrates a partially stained membrane 910. Illustratively, a compression of flow control mechanism 600 may be configured to apply a visualization fluid to an internal limiting membrane 460. In one or more embodiments, an application of a visualization fluid to an internal limiting membrane 460 may be configured to allow a surgeon to visualize a transparent membrane 900 as a partially stained membrane 910. Illustratively, internal limiting membrane 460 may comprise a partially stained membrane 910, e.g., when a visualization fluid is partially applied to internal limiting membrane 460. In one or more embodiments, internal limiting membrane 460 may comprise a partially stained membrane 910, e.g., when flow control mechanism 600 is partially compressed. Illustratively, a compression of flow control mechanism 600 may be configured to decrease a volume of visualization fluid chamber 610. In one or more embodiments, a decrease of a volume of visualization fluid chamber 610 may increase a pressure within visualization fluid chamber 610. Illustratively, an increase of a pressure within visualization fluid chamber 610 may be configured to increase a pressure within visualization fluid conduit 310. In one or more embodiments, an increase of a pressure within visualization fluid chamber 610 and visualization fluid conduit 310 may be configured to irrigate a visualization fluid out of visualization fluid chamber 610 and into visualization fluid conduit 310. Illustratively, an increase in a pressure within visualization fluid chamber 610 and visualization fluid conduit 310 may be configured to irrigate a visualization fluid out of visualization fluid conduit 310 and into hypodermic tube 220. In one or more embodiments, visualization fluid conduit distal taper 315 may be configured to facilitate an irrigation of a visualization fluid out of irrigation fluid conduit 310 and into hypodermic tube 220. Illustratively, an increase in a pressure within visualization fluid chamber 610 and visualization fluid conduit 310 may be configured to irrigate a visualization fluid out of hypodermic tube distal end 221 and onto internal limiting membrane 460. In one or more embodiments, a compression of flow control mechanism 600 may be configured to irrigate a visualization fluid out of hypodermic tube distal end 221 and onto internal limiting membrane 460. Illustratively, a compression of flow control mechanism 600 may be configured to irrigate a visualization fluid out of hypodermic tube distal end 221 at a flow rate in a range of 0.1 to 10.0 milliliters per minute. In one or more embodiments, a compression of flow control mechanism 600 may be configured to irrigate a visualization fluid out of hypodermic tube distal end 221 at a flow rate less than 0.1 milliliters per minute or greater than 10.0 milliliters per minute. Illustratively, a surgeon may selectively control a visualization fluid irrigation flow rate, e.g., by covering or exposing a portion of pressure vent 630. In one or more embodiments, a surgeon may selectively decrease a pressure within visualization fluid chamber 610, e.g., by exposing a portion of pressure vent 630. Illustratively, a surgeon may selectively decrease a visualization fluid irrigation flow rate, e.g., by exposing a portion of pressure vent 630. In one or more embodiments, a surgeon may selectively increase a pressure within visualization fluid chamber 610, e.g., by covering a portion of pressure vent 630. Illustratively, a surgeon may selectively increase a visualization fluid irrigation flow rate, e.g., by covering a portion of pressure vent 630. In one or more embodiments, a surgeon may selectively control a discrete volume of a visualization fluid irrigated out of hypodermic tube distal end 221, e.g., by covering or exposing a portion of pressure vent 630. Illustratively, a compression of flow control mechanism 600 may be configured to irrigate a visualization fluid out of hypodermic tube distal end 221 in discrete volumes, e.g., a compression of flow control mechanism 600 may be configured to irrigate a visualization fluid out of hypodermic tube distal end 221 in discrete drops. In one or more embodiments, a compression of flow control mechanism 600 may be configured to irrigate a visualization fluid out of hypodermic tube distal end 221 in discrete drops wherein each discrete drop has a volume in a range of 0.001 to 0.08 milliliters, e.g., each discrete drop may have a volume of 0.05 milliliters. Illustratively, a compression of flow control mechanism 600 may be configured to irrigate a visualization fluid out of hypodermic tube distal end 221 in discrete drops wherein each drop has a volume less than 0.001 milliliters or greater than 0.08 milliliters. In one or more embodiments, an application of a first compressive force to a portion of flow control mechanism 600 may be configured to irrigate a first drop of a visualization fluid out of hypodermic tube distal end 221. Illustratively, an application of a second compressive force to a portion of flow control mechanism 600 may be configured to irrigate a second drop of a visualization fluid out of hypodermic tube distal end 221. In one or more embodiments, an application of a third compressive force to a portion of flow control mechanism 600 may be configured to irrigate a third drop of a visualization fluid out of hypodermic tube distal end 221. Illustratively, the second compressive force may have a greater magnitude than the first compressive force and the third compressive force may have a greater magnitude than the second compressive force. In one or more embodiments, a compression of flow control mechanism 600 may be configured to apply a visualization fluid to an internal limiting membrane 460, e.g., a compression of flow control mechanism 600 may be configured to allow a surgeon to visualize a transparent membrane 900 as a partially stained membrane 910.

FIG. 9C illustrates a fully stained membrane 920. Illustratively, a compression of flow control mechanism 600 may be configured to apply a visualization fluid to an internal limiting membrane 460. In one or more embodiments, an application of a visualization fluid to an internal limiting membrane 460 may be configured to allow a surgeon to visualize a partially stained membrane 910 as a fully stained membrane 920. Illustratively, internal limiting membrane 460 may comprise a fully stained membrane 920, e.g., when a visualization fluid is fully applied to internal limiting membrane 460. In one or more embodiments, internal limiting membrane 460 may comprise a fully stained membrane 920, e.g., when flow control mechanism 600 is fully compressed. Illustratively, a compression of flow control mechanism 600 may be configured to decrease a volume of visualization fluid chamber 610. In one or more embodiments, a decrease of a volume of visualization fluid chamber 610 may increase a pressure within visualization fluid chamber 610. Illustratively, an increase of a pressure within visualization fluid chamber 610 may be configured to increase a pressure within visualization fluid conduit 310. In one or more embodiments, an increase of a pressure within visualization fluid chamber 610 and visualization fluid conduit 310 may be configured to irrigate a visualization fluid out of visualization fluid chamber 610 and into visualization fluid conduit 310. Illustratively, an increase in a pressure within visualization fluid chamber 610 and visualization fluid conduit 310 may be configured to irrigate a visualization fluid out of visualization fluid conduit 310 and into hypodermic tube 220. In one or more embodiments, visualization fluid conduit distal taper 315 may be configured to facilitate an irrigation of a visualization fluid out of irrigation fluid conduit 310 and into hypodermic tube 220. Illustratively, an increase in a pressure within visualization fluid chamber 610 and visualization fluid conduit 310 may be configured to irrigate a visualization fluid out of hypodermic tube distal end 221 and onto internal limiting membrane 460. In one or more embodiments, a compression of flow control mechanism 600 may be configured to irrigate a visualization fluid out of hypodermic tube distal end 221 and onto internal limiting membrane 460. Illustratively, a compression of flow control mechanism 600 may be configured to irrigate a visualization fluid out of hypodermic tube distal end 221 at a flow rate in a range of 0.1 to 10.0 milliliters per minute. In one or more embodiments, a compression of flow control mechanism 600 may be configured to irrigate a visualization fluid out of hypodermic tube distal end 221 at a flow rate less than 0.1 milliliters per minute or greater than 10.0 milliliters per minute. Illustratively, a surgeon may selectively control a visualization fluid irrigation flow rate, e.g., by covering or exposing a portion of pressure vent 630. In one or more embodiments, a surgeon may selectively decrease a pressure within visualization fluid chamber 610, e.g., by exposing a portion of pressure vent 630. Illustratively, a surgeon may selectively decrease a visualization fluid irrigation flow rate, e.g., by exposing a portion of pressure vent 630. In one or more embodiments, a surgeon may selectively increase a pressure within visualization fluid chamber 610, e.g., by covering a portion of pressure vent 630. Illustratively, a surgeon may selectively increase a visualization fluid irrigation flow rate, e.g., by covering a portion of pressure vent 630. In one or more embodiments, a surgeon may selectively control a discrete volume of a visualization fluid irrigated out of hypodermic tube distal end 221, e.g., by covering or exposing a portion of pressure vent 630. Illustratively, a compression of flow control mechanism 600 may be configured to irrigate a visualization fluid out of hypodermic tube distal end 221 in discrete volumes, e.g., a compression of flow control mechanism 600 may be configured to irrigate a visualization fluid out of hypodermic tube distal end 221 in discrete drops. In one or more embodiments, a compression of flow control mechanism 600 may be configured to irrigate a visualization fluid out of hypodermic tube distal end 221 in discrete drops wherein each discrete drop has a volume in a range of 0.001 to 0.08 milliliters, e.g., each discrete drop may have a volume of 0.05 milliliters. Illustratively, a compression of flow control mechanism 600 may be configured to irrigate a visualization fluid out of hypodermic tube distal end 221 in discrete drops wherein each drop has a volume less than 0.001 milliliters or greater than 0.08 milliliters. In one or more embodiments, an application of a first compressive force to a portion of flow control mechanism 600 may be configured to irrigate a first drop of a visualization fluid out of hypodermic tube distal end 221. Illustratively, an application of a second compressive force to a portion of flow control mechanism 600 may be configured to irrigate a second drop of a visualization fluid out of hypodermic tube distal end 221. In one or more embodiments, an application of a third compressive force to a portion of flow control mechanism 600 may be configured to irrigate a third drop of a visualization fluid out of hypodermic tube distal end 221. Illustratively, the second compressive force may have a greater magnitude than the first compressive force and the third compressive force may have a greater magnitude than the second compressive force. In one or more embodiments, a compression of flow control mechanism 600 may be configured to apply a visualization fluid to an internal limiting membrane 460, e.g., a compression of flow control mechanism 600 may be configured to allow a surgeon to visualize a partially stained membrane 910 as a fully stained membrane 920.

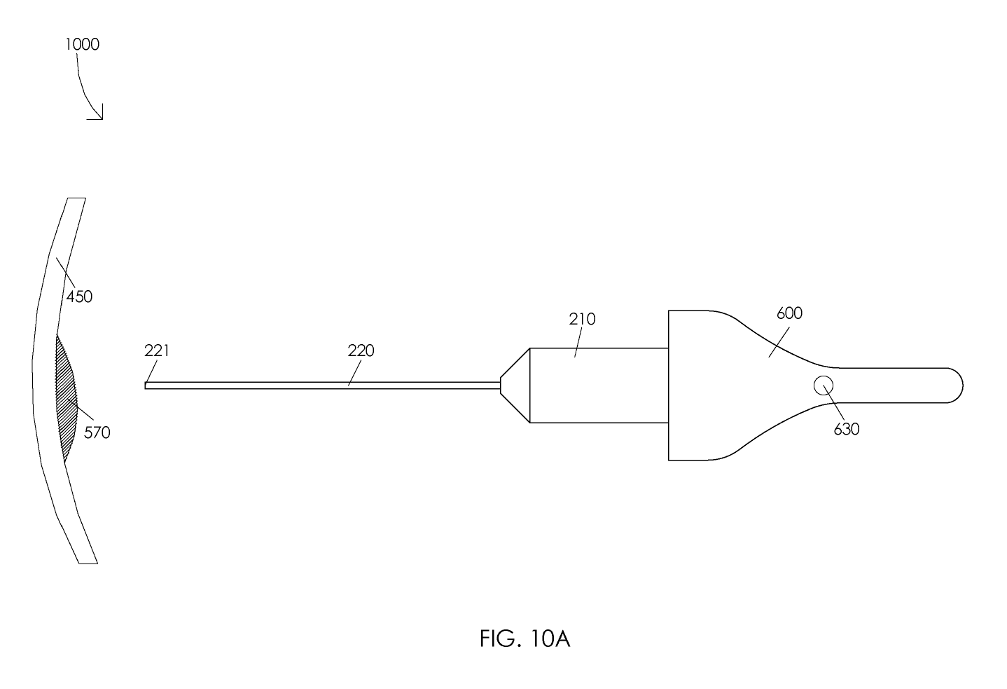

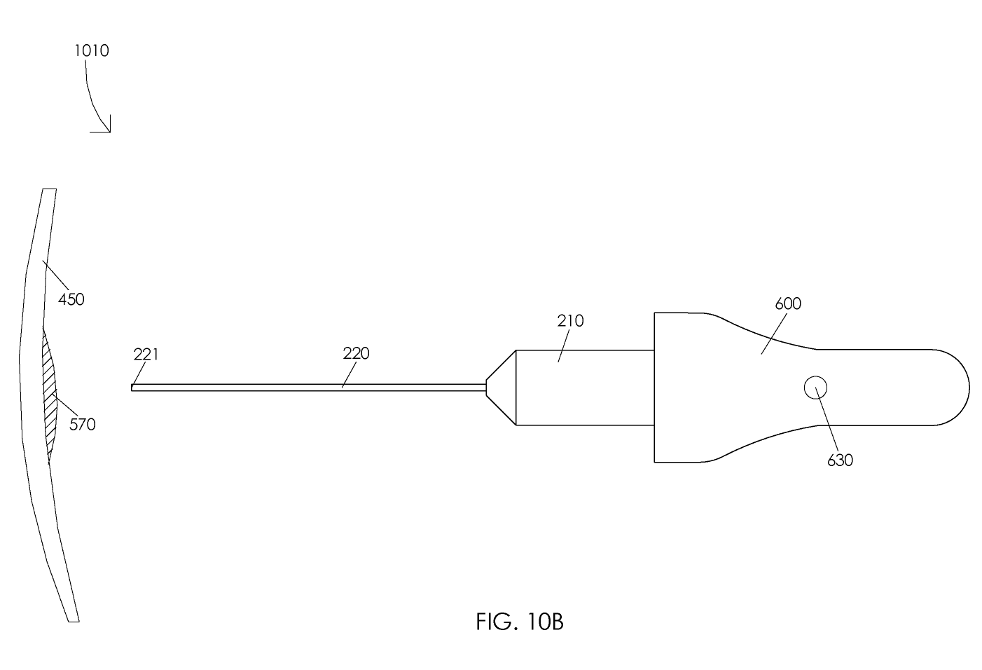

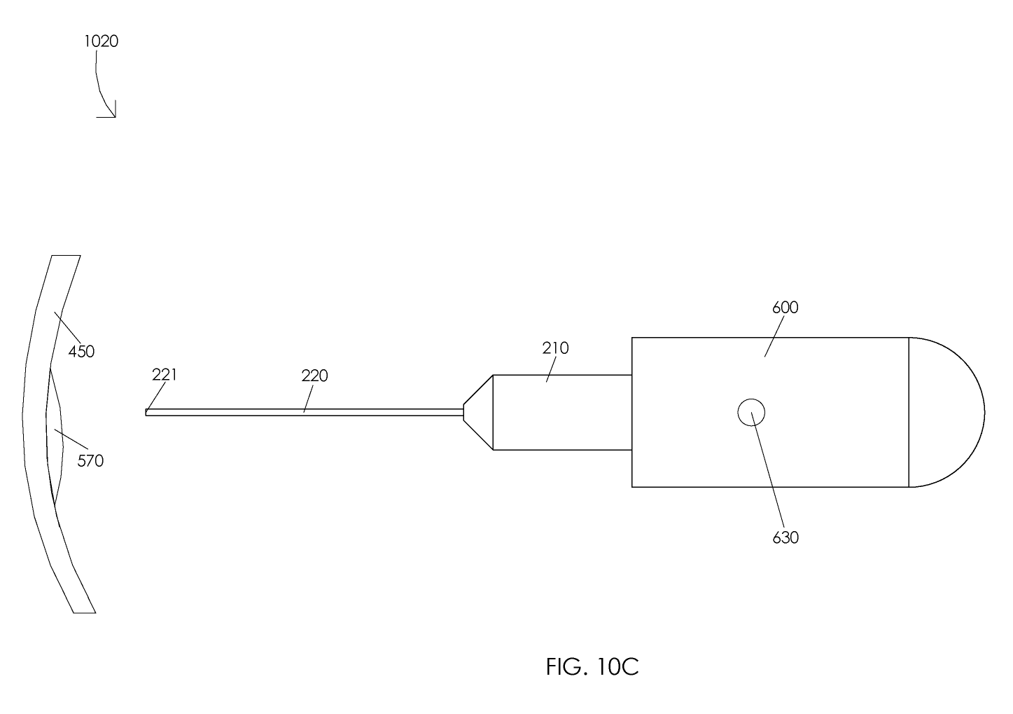

FIGS. 10A, 10B, and 10C are schematic diagrams illustrating a removal of a visualization fluid from a surgical site 570. FIG. 10A illustrates a surgical site with excess visualization fluid 1000. In one or more embodiments, a visualization fluid, e.g., indocyanine green dye, kenalog, trypan blue dye, etc., may be at surgical site 570. Illustratively, surgical site 570 may comprise a surgical site with excess visualization fluid 1000, e.g., when flow control mechanism 600 is fully compressed. In one or more embodiments, surgical site 570 may comprise a surgical site with excess visualization fluid 1000, e.g., after an application of a visualization fluid to an internal limiting membrane 460. Illustratively, surgical site 570 may comprise a surgical site with excess visualization fluid 1000, e.g., when pressure vent 630 is fully covered. In one or more embodiments, a surgical site with excess visualization fluid 1000 may be adjacent to a retinal tissue 450, e.g., a surgical site with excess visualization fluid 1000 may be adjacent to an internal limiting membrane 460.

FIG. 10B illustrates a surgical site with excess visualization fluid partially removed 1010. Illustratively, a decompression of flow control mechanism 600 may be configured to remove a visualization fluid from a surgical site 570. In one or more embodiments, a removal of a visualization fluid from a surgical site 570 may be configured to allow a surgeon to visualize a surgical site with excess visualization fluid 1000 as a surgical site with excess visualization fluid partially removed 1010. Illustratively, surgical site 570 may comprise a surgical site with excess visualization fluid partially removed 1010, e.g., when a visualization fluid is partially removed from surgical site 570. In one or more embodiments, surgical site 570 may comprise a surgical site with excess visualization fluid partially removed 1010, e.g., when a portion of pressure vent 630 is exposed. Illustratively, a surgeon may expose a portion of pressure vent 630, e.g., by uncovering a portion of pressure vent 630. In one or more embodiments, exposing a portion of pressure vent 630 may be configured to decrease a pressure within visualization fluid chamber 610. Illustratively, a decrease of a pressure within visualization fluid chamber 610 may be configured to decrease a pressure within visualization fluid conduit 310. In one or more embodiments, a decrease of a pressure within visualization fluid chamber 610 and visualization fluid conduit 310 may be configured to aspirate a visualization fluid out of surgical site 570 and into hypodermic tube 220. Illustratively, an intraocular pressure of an eye may be configured to aspirate a visualization fluid out of surgical site 570 and into hypodermic tube distal end 221. In one or more embodiments, surgical site 570 may comprise a surgical site with excess visualization fluid partially removed 1010, e.g., when flow control mechanism 600 is partially decompressed. Illustratively, a decompression of flow control mechanism 600 may be configured to increase a volume of visualization fluid chamber 610. In one or more embodiments, an increase of a volume of visualization fluid chamber 610 may decrease a pressure within visualization fluid chamber 610. Illustratively, a decrease of a pressure within visualization fluid chamber 610 may be configured to decrease a pressure within visualization fluid conduit 310. In one or more embodiments, a decrease of a pressure within visualization fluid chamber 610 and visualization fluid conduit 310 may be configured to aspirate a visualization fluid out of surgical site 570 and into hypodermic tube 220. For example, a decrease of a pressure within visualization fluid chamber 610 and visualization fluid conduit 310 may be configured to aspirate a visualization fluid out of surgical site 570 and into hypodermic tube distal end 221. Illustratively, a decrease of a pressure within visualization fluid chamber 610 and visualization fluid conduit 310 may be configured to aspirate a visualization fluid through hypodermic tube 220 and out of hypodermic tube proximal end 222. In one or more embodiments, a decrease of a pressure within visualization fluid chamber 610 and visualization fluid conduit 310 may be configured to aspirate a visualization fluid out of hypodermic tube proximal end 222 and into visualization fluid conduit 310. Illustratively, a decrease of a pressure within visualization fluid chamber 610 and visualization fluid conduit 310 may be configured to aspirate a visualization fluid out of visualization fluid conduit 310 and into visualization fluid chamber 610. In one or more embodiments, a surgeon may selectively control a visualization fluid aspiration flow rate, e.g., by covering or exposing a portion of pressure vent 630. Illustratively, a surgeon may selectively decrease a pressure within visualization fluid chamber 610, e.g., by exposing a portion of pressure vent 630. In one or more embodiments, a surgeon may selectively increase a visualization fluid aspiration flow rate, e.g., by exposing a portion of pressure vent 630. Illustratively, a surgeon may selectively increase a pressure within visualization fluid chamber 610, e.g., by covering a portion of pressure vent 630. In one or more embodiments, a surgeon may selectively decrease a visualization fluid aspiration flow rate, e.g., by covering a portion of pressure vent 630. Illustratively, a decompression of flow control mechanism 600 may be configured to aspirate a visualization fluid into hypodermic tube distal end 221 at a flow rate in a range of 0.06 to 41.0 milliliters per minute. In one or more embodiments, a decompression of flow control mechanism 600 may be configured to aspirate a visualization fluid into hypodermic tube distal end 221 at a flow rate less than 0.06 milliliters per minute or greater than 41.0 milliliters per minute. Illustratively, a decompression of flow control mechanism 600 may be configured to remove a visualization fluid from a surgical site 570, e.g., a decompression of flow control mechanism 600 may be configured to allow a surgeon to visualize a surgical site with excess visualization fluid 1000 as a surgical site with excess visualization fluid partially removed 1010.

FIG. 10C illustrates a surgical site with excess visualization fluid completely removed 1020. Illustratively, a decompression of flow control mechanism 600 may be configured to remove a visualization fluid from a surgical site 570. In one or more embodiments, a removal of a visualization fluid from a surgical site 570 may be configured to allow a surgeon to visualize a surgical site with excess visualization fluid partially removed 1010 as a surgical site with excess visualization fluid completely removed 1020. Illustratively, surgical site 570 may comprise a surgical site with excess visualization fluid completely removed 1020, e.g., when a visualization fluid is completely removed from surgical site 570. In one or more embodiments, surgical site 570 may comprise a surgical site with excess visualization fluid completely removed 1020, e.g., when a portion of pressure vent 630 is exposed. Illustratively, a surgeon may expose a portion of pressure vent 630, e.g., by uncovering a portion of pressure vent 630. In one or more embodiments, exposing a portion of pressure vent 630 may be configured to decrease a pressure within visualization fluid chamber 610. Illustratively, a decrease of a pressure within visualization fluid chamber 610 may be configured to decrease a pressure within visualization fluid conduit 310. In one or more embodiments, a decrease of a pressure within visualization fluid chamber 610 and visualization fluid conduit 310 may be configured to aspirate a visualization fluid out of surgical site 570 and into hypodermic tube 220. Illustratively, an intraocular pressure of an eye may be configured to aspirate a visualization fluid out of surgical site 570 and into hypodermic tube distal end 221. In one or more embodiments, surgical site 570 may comprise a surgical site with excess visualization fluid completely removed 1020, e.g., when flow control mechanism 600 is fully decompressed. Illustratively, a decompression of flow control mechanism 600 may be configured to increase a volume of visualization fluid chamber 610. In one or more embodiments, an increase of a volume of visualization fluid chamber 610 may decrease a pressure within visualization fluid chamber 610. Illustratively, a decrease of a pressure within visualization fluid chamber 610 may be configured to decrease a pressure within visualization fluid conduit 310. In one or more embodiments, a decrease of a pressure within visualization fluid chamber 610 and visualization fluid conduit 310 may be configured to aspirate a visualization fluid out of surgical site 570 and into hypodermic tube 220. For example, a decrease of a pressure within visualization fluid chamber 610 and visualization fluid conduit 310 may be configured to aspirate a visualization fluid out of surgical site 570 and into hypodermic tube distal end 221. Illustratively, a decrease of a pressure within visualization fluid chamber 610 and visualization fluid conduit 310 may be configured to aspirate a visualization fluid through hypodermic tube 220 and out of hypodermic tube proximal end 222. In one or more embodiments, a decrease of a pressure within visualization fluid chamber 610 and visualization fluid conduit 310 may be configured to aspirate a visualization fluid out of hypodermic tube proximal end 222 and into visualization fluid conduit 310. Illustratively, a decrease of a pressure within visualization fluid chamber 610 and visualization fluid conduit 310 may be configured to aspirate a visualization fluid out of visualization fluid conduit 310 and into visualization fluid chamber 610. In one or more embodiments, a surgeon may selectively control a visualization fluid aspiration flow rate, e.g., by covering or exposing a portion of pressure vent 630. Illustratively, a surgeon may selectively decrease a pressure within visualization fluid chamber 610, e.g., by exposing a portion of pressure vent 630. In one or more embodiments, a surgeon may selectively increase a visualization fluid aspiration flow rate, e.g., by exposing a portion of pressure vent 630. Illustratively, a surgeon may selectively increase a pressure within visualization fluid chamber 610, e.g., by covering a portion of pressure vent 630. In one or more embodiments, a surgeon may selectively decrease a visualization fluid aspiration flow rate, e.g., by covering a portion of pressure vent 630. Illustratively, a decompression of flow control mechanism 600 may be configured to aspirate a visualization fluid into hypodermic tube distal end 221 at a flow rate in a range of 0.06 to 41.0 milliliters per minute. In one or more embodiments, a decompression of flow control mechanism 600 may be configured to aspirate a visualization fluid into hypodermic tube distal end 221 at a flow rate less than 0.06 milliliters per minute or greater than 41.0 milliliters per minute. Illustratively, a decompression of flow control mechanism 600 may be configured to remove a visualization fluid from a surgical site 570, e.g., a decompression of flow control mechanism 600 may be configured to allow a surgeon to visualize a surgical site with excess visualization fluid partially removed 1010 as a surgical site with excess visualization fluid completely removed 1020.

Illustratively, one or more properties of a membrane visualization instrument may be adjusted to attain one or more desired membrane visualization instrument features. In one or more embodiments, membrane visualization instrument may be configured to apply a visualization fluid to an epiretinal membrane, e.g., a surgeon may compress flow control mechanism 600 to irrigate a visualization fluid out of hypodermic tube distal end 221 and onto an epiretinal membrane. Illustratively, an epiretinal membrane may comprise a transparent membrane 900, e.g., when flow control mechanism 600 is fully decompressed. In one or more embodiments, a compression of flow control mechanism 600 may be configured to irrigate a visualization fluid out of hypodermic tube distal end 221 and onto an epiretinal membrane. Illustratively, an application of a visualization fluid to an epiretinal membrane may be configured to allow a surgeon to visualize a transparent membrane 900 as a partially stained membrane 910. In one or more embodiments, an epiretinal membrane may comprise a partially stained membrane 910, e.g., when a visualization fluid is partially applied to the epiretinal membrane. Illustratively, compression of flow control mechanism 600 may be configured to irrigate a visualization fluid out of hypodermic tube distal end 221 and onto an epiretinal membrane. In one or more embodiments, an application of a visualization fluid to an epiretinal membrane may be configured to allow a surgeon to visualize a partially stained membrane 910 as a fully stained membrane 920. Illustratively, an epiretinal membrane may comprise a fully stained membrane 920, e.g., when a visualization fluid is fully applied to the epiretinal membrane.

In one or more embodiments, a volume of visualization fluid chamber 910 may be adjusted, e.g., to vary an amount of a visualization fluid available for applying to internal limiting membrane 460 or an epiretinal membrane. Illustratively, a stiffness of flow control mechanism 600 may be adjusted, e.g., to vary a magnitude of a compressive force configured to irrigate a particular volume of a visualization fluid out of hypodermic tube distal end 221. In one or more embodiments, flow control mechanism 600 may comprise a transparent material configured to allow a surgeon to visualize an amount of a visualization fluid disposed within visualization fluid chamber 610, e.g., flow control mechanism 600 may comprise a transparent silicon material. Illustratively, an inner diameter of hypodermic tube 220 may be adjusted, e.g., to vary a visualization fluid irrigation flow rate. In one or more embodiments, an inner diameter of hypodermic tube 220 may be adjusted, e.g., to vary a visualization fluid aspiration flow rate. Illustratively, hypodermic tube 220 may have an inner diameter in a range of 0.009 to 0.031 inches. In one or more embodiments, hypodermic tube 220 may have an inner diameter less than 0.009 inches or greater than 0.031 inches. Illustratively, flow control mechanism 600 may comprise a plurality of pressure vents 630. In one or more embodiments, flow control mechanism 600 may comprise a slight indentation surrounding pressure vent 630, e.g., a slight indentation may be configured to facilitate a covering of a portion of pressure vent 630 during a surgical procedure.

Illustratively, a visualization fluid, e.g., indocyanine green dye, kenalog, trypan blue dye, etc., may be disposed within visualization fluid chamber 610. In one or more embodiments, a visualization fluid may be disposed within a hermetically sealed capsule, e.g., a visualization fluid may be disposed within a hermetically sealed pouch. Illustratively, the hermetically sealed capsule may be disposed within visualization fluid chamber 610, e.g., the hermetically sealed capsule may be disposed between visualization fluid guide proximal end 212 and flow control mechanism proximal end 602. In one or more embodiments, a portion of the hermetically sealed capsule may be disposed within visualization fluid conduit 310, e.g., the hermetically sealed capsule may be disposed between visualization fluid guide distal end 211 and flow control mechanism proximal end 602. Illustratively, a compression of flow control mechanism 600 may be configured to rupture the hermetically sealed capsule and release a visualization fluid within visualization fluid chamber 610. In one or more embodiments, a first compression of flow control mechanism 600 may be configured to release a visualization fluid within visualization fluid chamber 610 and a second compression of flow control mechanism 600 may be configured to irrigate the visualization fluid through hypodermic tube 220 and out of hypodermic tube distal end 221.

Illustratively, a visualization fluid may comprise two or more component fluids, e.g., two or more component fluids may be combined. In one or more embodiments, a first component fluid may be disposed within a first hermetically sealed capsule and a second component fluid may be disposed within a second hermetically sealed capsule. Illustratively, the first hermetically sealed capsule and the second hermetically sealed capsule may be disposed within visualization fluid chamber 610. In one or more embodiments, a compression of flow control mechanism 600 may be configured to rupture the first hermetically sealed capsule and the second hermetically sealed capsule. Illustratively, a rupture of the first hermetically sealed capsule and the second hermetically sealed capsule may release the first component fluid and the second component fluid within visualization fluid chamber 610 wherein the first component fluid and the second component fluid are combined. In one or more embodiments, a first compression of flow control mechanism 600 may be configured to release a first fluid component and a second fluid component within visualization fluid chamber 610 and a second compression of flow control mechanism 600 may be configured to irrigate a visualization fluid through hypodermic tube 220 and out of hypodermic tube distal end 221.

Illustratively, a visualization fluid, e.g., indocyanine green dye, kenalog, trypan blue dye, etc., may be external to a membrane visualization instrument, e.g., a visualization fluid may be disposed within a container in an operating room. In one or more embodiments, a surgeon or a surgeon's assistant may decompress flow control mechanism 600, e.g., to aspirate a visualization fluid out of a container and into hypodermic tube distal end 221. Illustratively, a surgeon or a surgeon's assistant may decompress flow control mechanism 600, e.g., to aspirate a visualization fluid out of hypodermic tube proximal end 222 and into visualization fluid conduit 310. In one or more embodiments, a surgeon or a surgeon's assistant may decompress flow control mechanism 600, e.g., to aspirate a visualization fluid out of visualization fluid conduit 310 and into visualization fluid chamber 610. Illustratively, a surgeon may compress flow control mechanism 600, e.g., to apply a visualization fluid to a transparent membrane 900.

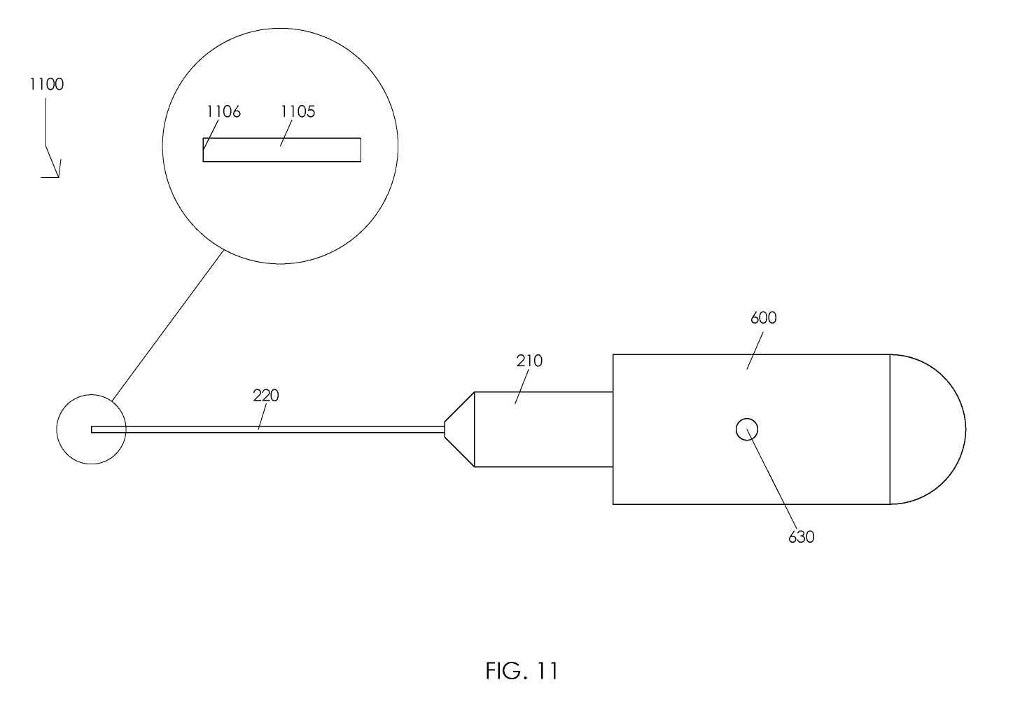

FIG. 11 is a schematic diagram illustrating a membrane visualization instrument with a blunt tip 1100. Illustratively, a membrane visualization instrument with a blunt tip 1100 may comprise a flow control mechanism 600, a visualization fluid guide 210, and a hypodermic tube 220. In one or more embodiments, a portion of hypodermic tube 220 may comprise a blunt tip 1105 having a blunt tip distal end 1106, e.g., hypodermic tube distal end 221 may comprise blunt tip distal end 1106. Illustratively, a compression of flow control mechanism 600 may be configured to irrigate a visualization fluid out of blunt tip distal end 1106 at a flow rate in a range of 2.5 to 10.0 milliliters per minute. In one or more embodiments, a compression of flow control mechanism 600 may be configured to irrigate a visualization fluid out of blunt tip distal end 1106 at a flow rate less than 2.5 milliliters per minute or greater than 10.0 milliliters per minute. Illustratively, a decompression of flow control mechanism 600 may be configured to aspirate a visualization fluid into blunt tip distal end 1106 at a flow rate in a range of 2.5 to 40.0 milliliters per minute. In one or more embodiments, a decompression of flow control mechanism 600 may be configured to aspirate a visualization fluid into blunt tip distal end 1106 at a flow rate less than 2.5 milliliters per minute or greater than 40.0 milliliters per minute.

Illustratively, a membrane visualization instrument with a blunt tip 1100 may be configured to perform an air-fluid exchange procedure. In one or more embodiments, an air infusion pump may be inserted through a first incision in an eye, e.g., an air infusion pump may be configured to maintain an intraocular pressure during a surgical procedure. Illustratively, a surgeon may perform an air-fluid exchange by covering pressure vent 630 and inserting blunt tip distal end 1106 into a second incision in the eye. In one or more embodiments, after inserting blunt tip 1105 into the eye, the surgeon may expose a portion of pressure vent 630. Illustratively, exposing a portion of pressure vent 630 may be configured to decrease a pressure within visualization fluid chamber 610. In one or more embodiments, a decrease of a pressure within visualization fluid chamber 610 may be configured to decrease a pressure within visualization fluid conduit 310 and hypodermic tube 220. Illustratively, a decrease of a pressure within visualization fluid conduit 310 and hypodermic tube 220 may be configured to aspirate a vitreous out of the eye and into hypodermic tube 220 through blunt tip distal end 1106. In one or more embodiments, a decrease of a pressure within visualization fluid conduit 310 and hypodermic tube 220 may be configured to aspirate a vitreous out of hypodermic tube proximal end 222 and into visualization fluid conduit 310. Illustratively, a decrease of a pressure within visualization fluid chamber 610 may be configured to aspirate a vitreous out of visualization fluid conduit 310 and into visualization fluid chamber 610. In one or more embodiments, as a vitreous is aspirated out of an eye, an air infusion pump may be configured to replace the vitreous with a gas, e.g., air. Illustratively, the gas may be configured to form a gas bubble inside of an eye. In one or more embodiments, the gas bubble may be configured to close and seal a macular hole, seal a retinal tear, facilitate reattachment of a detached retina, etc.

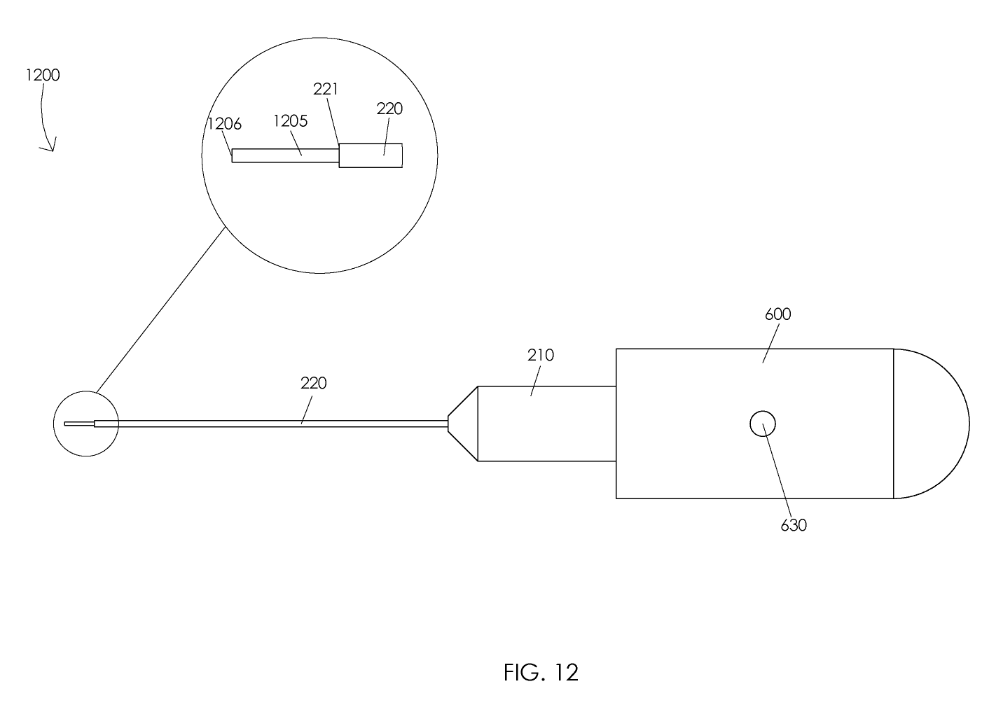

FIG. 12 is a schematic diagram illustrating a membrane visualization instrument with a soft tip 1200. Illustratively, a membrane visualization instrument with a soft tip 1200 may comprise a flow control mechanism 600, a visualization fluid guide 210, a hypodermic tube 220, and a soft tip 1205. In one or more embodiments, soft tip 1205 may have a stiffness configured to contact a retinal tissue 450 without damaging the retinal tissue 450. Illustratively, soft tip 1205 may be configured to manipulate a portion of a retinal tissue 450. In one or more embodiments, soft tip 1205 may be configured to be inserted into a retinal tear, e.g., to drain a subretinal fluid. Illustratively, soft tip 1205 may comprise a silicon tube, e.g., configured to irrigate and aspirate a visualization fluid. In one or more embodiments, a portion of soft tip 1205 may be disposed within hypodermic tube 220, e.g., a proximal end of soft tip 1205 may be disposed within hypodermic tube 220. Illustratively, a portion of soft tip 1205 may be fixed within hypodermic tube 220, e.g., by an adhesive or any suitable fixation means. In one or more embodiments, soft tip 1205 may be fixed within hypodermic tube 220 wherein a soft tip distal end 1206 extends from hypodermic tube distal end 221. Illustratively, a compression of flow control mechanism 600 may be configured to irrigate a visualization fluid out of visualization fluid conduit 310 and into hypodermic tube proximal end 222. In one or more embodiments, a compression of flow control mechanism 600 may be configured to irrigate a visualization fluid out of hypodermic tube 220 and into soft tip 1205. Illustratively, a compression of flow control mechanism 600 may be configured to irrigate a visualization fluid out of soft tip distal end 1206. In one or more embodiments, a compression of flow control mechanism 600 may be configured to irrigate a visualization fluid out of soft tip distal end 1206 at a flow rate in a range of 0.1 to 4.5 milliliters per minute. Illustratively, a compression of flow control mechanism 600 may be configured to irrigate a visualization fluid out of soft tip distal end 1206 at a flow rate less than 0.1 milliliters per minute or greater than 4.5 milliliters per minute.

In one or more embodiments, a decompression of flow control mechanism 600 may be configured to aspirate a visualization fluid into soft tip distal end 1206. Illustratively, a decompression of flow control mechanism 600 may be configured to aspirate a visualization fluid out of soft tip 1205 and into hypodermic tube 220. In one or more embodiments, a decompression of flow control mechanism 600 may be configured to aspirate a visualization fluid out of hypodermic tube distal end 220 and into visualization fluid conduit 310. Illustratively, a decompression of flow control mechanism 600 may be configured to aspirate a visualization fluid into soft tip distal end 1206 at a flow rate in a range of 0.06 to 13.5 milliliters per minute. In one or more embodiments, a decompression of flow control mechanism 600 may be configured to aspirate a visualization fluid into soft tip distal end 1206 at a flow rate less than 0.06 milliliters per minute or greater than 13.5 milliliters per minute.

Illustratively, a membrane visualization instrument with a soft tip 1200 may be configured to perform an air-fluid exchange procedure. In one or more embodiments, an air infusion pump may be inserted through a first incision in an eye, e.g., an air infusion pump may be configured to maintain an intraocular pressure during a surgical procedure. Illustratively, a surgeon may perform an air-fluid exchange by covering pressure vent 630 and inserting soft tip distal end 1206 into a second incision in the eye. In one or more embodiments, after inserting soft tip 1205 into the eye, the surgeon may expose a portion of pressure vent 630. Illustratively, exposing a portion of pressure vent 630 may be configured to decrease a pressure within visualization fluid chamber 610. In one or more embodiments, a decrease of a pressure within visualization fluid chamber 610 may be configured to decrease a pressure within visualization fluid conduit 310 and hypodermic tube 220. Illustratively, a decrease of a pressure within visualization fluid conduit 310 and hypodermic tube 220 may be configured to aspirate a vitreous out of the eye and into soft tip distal end 1206. In one or more embodiments, a decrease of a pressure within visualization fluid conduit 310 and hypodermic tube 220 may be configured to aspirate a vitreous out of soft tip 1205 and into hypodermic tube 220. Illustratively, a decrease of a pressure within visualization fluid conduit 310 may be configured to aspirate a vitreous out of hypodermic tube proximal end 222 and into visualization fluid conduit 310. In one or more embodiments, a decrease of a pressure within visualization fluid chamber 610 may be configured to aspirate a vitreous out of visualization fluid conduit 310 and into visualization fluid chamber 610. Illustratively, as a vitreous is aspirated out of an eye, an air infusion pump may be configured to replace the vitreous with a gas, e.g., air. In one or more embodiments, the gas may be configured to form a gas bubble inside of an eye. Illustratively, the gas bubble may be configured to close and seal a macular hole, seal a retinal tear, facilitate reattachment of a detached retina, etc.

In one or more embodiments, a membrane visualization instrument with a soft tip 1200 may be configured to perform a subretinal fluid drainage procedure. Illustratively, a surgeon may perform a subretinal fluid drainage by covering pressure vent 630 and inserting soft tip distal end 1206 into a second incision in the eye. In one or more embodiments, a surgeon may insert soft tip distal end 1206 into a retinal tear and then expose a portion of pressure vent 630. Illustratively, exposing a portion of pressure vent 630 may be configured to decrease a pressure within visualization fluid chamber 610 and visualization fluid conduit 310. In one or more embodiments, a decrease of a pressure within visualization fluid chamber 610 and visualization fluid conduit 310 may be configured to aspirate a subretinal fluid out of the eye and into soft tip distal end 1206. Illustratively, a decrease of a pressure within visualization fluid chamber 610 and visualization fluid conduit 310 may be configured to aspirate a subretinal fluid out of soft tip 1205 and into hypodermic tube 220. Illustratively, a decrease of a pressure within visualization fluid chamber 610 may be configured to aspirate a subretinal fluid out of hypodermic tube proximal end 222 and into visualization fluid conduit 310. In one or more embodiments, a decrease of a pressure within visualization fluid chamber 610 may be configured to aspirate a subretinal fluid out of visualization fluid conduit 310 and into visualization fluid chamber 610.

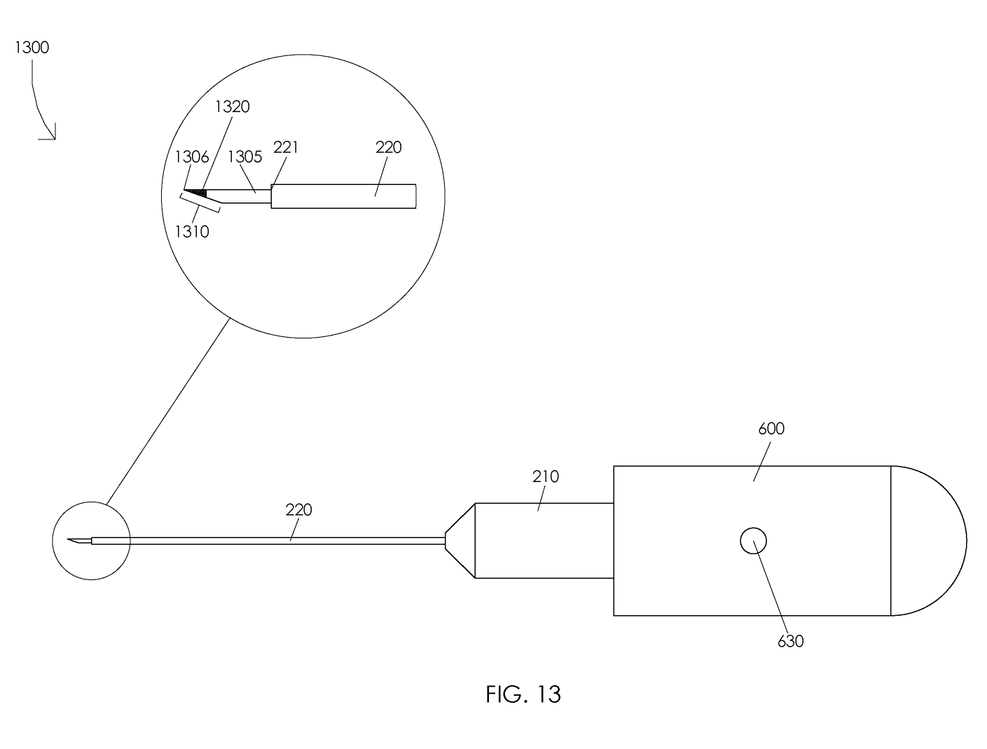

FIG. 13 is a schematic diagram illustrating a membrane visualization instrument for removing membranes 1300. Illustratively, a membrane visualization instrument for removing membranes 1300 may comprise a flow control mechanism 600, a visualization fluid guide 210, a hypodermic tube 220, and a flexible tip 1305. In one or more embodiments, flexible tip 1305 may comprise a tapered portion 1310 and an abrasive portion 1320. Illustratively, abrasive portion 1320 may be configured to facilitate removal of an internal limiting membrane 460 from retinal tissue 450. In one or more embodiments, abrasive portion 1320 may comprise inert particles, e.g., diamond particles, fixed to flexible tip 1305 by a biocompatible adhesive. Illustratively, flexible tip 1305 may comprise a silicon tube, e.g., configured to irrigate and aspirate a visualization fluid. In one or more embodiments, a portion of flexible tip 1305 may be disposed within hypodermic tube 220, e.g., a proximal end of flexible tip 1305 may be disposed within hypodermic tube 220. Illustratively, a portion of flexible tip 1305 may be fixed within hypodermic tube 220, e.g., by an adhesive or any suitable fixation means. In one or more embodiments, flexible tip 1305 may be fixed within hypodermic tube 220 wherein a flexible tip distal end 1306 extends from hypodermic tube distal end 221.

Illustratively, a compression of flow control mechanism 600 may be configured to irrigate a visualization fluid out of visualization fluid conduit 310 and into hypodermic tube proximal end 222. In one or more embodiments, a compression of flow control mechanism 600 may be configured to irrigate a visualization fluid out of hypodermic tube 220 and into flexible tip 1305. Illustratively, a compression of flow control mechanism 600 may be configured to irrigate a visualization fluid out of flexible tip distal end 1306. In one or more embodiments, a compression of flow control mechanism 600 may be configured to irrigate a visualization fluid out of flexible tip distal end 1306 at a flow rate in a range of 0.1 to 4.5 milliliters per minute. Illustratively, a compression of flow control mechanism 600 may be configured to irrigate a visualization fluid out of flexible tip distal end 1306 at a flow rate less than 0.1 milliliters per minute or greater than 4.5 milliliters per minute.

In one or more embodiments, a decompression of flow control mechanism 600 may be configured to aspirate a visualization fluid into flexible tip distal end 1306. Illustratively, a decompression of flow control mechanism 600 may be configured to aspirate a visualization fluid out of flexible tip 1305 and into hypodermic tube 220. In one or more embodiments, a decompression of flow control mechanism 600 may be configured to aspirate a visualization fluid out of hypodermic tube distal end 220 and into visualization fluid conduit 310. Illustratively, a decompression of flow control mechanism 600 may be configured to aspirate a visualization fluid into flexible tip distal end 1306 at a flow rate in a range of 0.06 to 13.5 milliliters per minute. In one or more embodiments, a decompression of flow control mechanism 600 may be configured to aspirate a visualization fluid into flexible tip distal end 1306 at a flow rate less than 0.06 milliliters per minute or greater than 13.5 milliliters per minute.

Illustratively, a membrane visualization instrument for removing membranes 1300 may be configured to perform an air-fluid exchange procedure. In one or more embodiments, an air infusion pump may be inserted through a first incision in an eye, e.g., an air infusion pump may be configured to maintain an intraocular pressure during a surgical procedure. Illustratively, a surgeon may perform an air-fluid exchange by covering pressure vent 630 and inserting flexible tip distal end 1306 into a second incision in the eye. In one or more embodiments, after inserting flexible tip 1305 into the eye, the surgeon may expose a portion of pressure vent 630. Illustratively, exposing a portion of pressure vent 630 may be configured to decrease a pressure within visualization fluid chamber 610. In one or more embodiments, a decrease of a pressure within visualization fluid chamber 610 may be configured to decrease a pressure within visualization fluid conduit 310 and hypodermic tube 220. Illustratively, a decrease of a pressure within visualization fluid conduit 310 and hypodermic tube 220 may be configured to aspirate a vitreous out of the eye and into flexible tip distal end 1306. In one or more embodiments, a decrease of a pressure within visualization fluid conduit 310 and hypodermic tube 220 may be configured to aspirate a vitreous out of flexible tip 1305 and into hypodermic tube 220. Illustratively, a decrease of a pressure within visualization fluid conduit 310 may be configured to aspirate a vitreous out of hypodermic tube proximal end 222 and into visualization fluid conduit 310. In one or more embodiments, a decrease of a pressure within visualization fluid chamber 610 may be configured to aspirate a vitreous out of visualization fluid conduit 310 and into visualization fluid chamber 610. Illustratively, as a vitreous is aspirated out of an eye, an air infusion pump may be configured to replace the vitreous with a gas, e.g., air. In one or more embodiments, the gas may be configured to form a gas bubble inside of an eye. Illustratively, the gas bubble may be configured to close and seal a macular hole, seal a retinal tear, facilitate reattachment of a detached retina, etc.

In one or more embodiments, a membrane visualization instrument for removing membranes 1300 may be configured to remove internal limiting membrane 460 from retinal tissue 450. Illustratively, a surgeon may remove an internal limiting membrane 460 from retinal tissue 450, e.g., by applying a visualization fluid to a transparent membrane 900. In one or more embodiments, an application of a visualization fluid to a transparent membrane 900 may be configured to allow the surgeon to visualize internal limiting membrane 460 as a fully stained membrane 920. Illustratively, the surgeon may manipulate flexible tip 1305 to remove a portion of internal limiting membrane 460 from retinal tissue 450, e.g., the surgeon may maneuver abrasive portion 1320 over internal limiting membrane 460 to separate a portion of internal limiting membrane 460 from retinal tissue 450. In one or more embodiments, the surgeon may remove additional portions of internal limiting membrane 460 from retinal tissue 450, e.g., by manipulating flexible tip 1305. Illustratively, the surgeon may manipulate flexible tip 1305 to separate a portion of internal limiting membrane 460 from retinal tissue 450, e.g., the surgeon may maneuver abrasive portion 1320 over internal limiting membrane 460 to raise a portion of internal limiting membrane 460 from retinal tissue 450. In one or more embodiments, a surgeon may grasp a raised portion of internal limiting membrane 460, e.g., with a forceps, and peel internal limiting membrane 460 from retinal tissue 450.

In one or more embodiments, a membrane visualization instrument for removing membranes 1300 may be configured to remove an epiretinal membrane. Illustratively, a surgeon may remove an epiretinal membrane, e.g., by applying a visualization fluid to a transparent membrane 900. In one or more embodiments, an application of a visualization fluid to a transparent membrane 900 may be configured to allow the surgeon to visualize an epiretinal membrane as a fully stained membrane 920. Illustratively, the surgeon may manipulate flexible tip 1305 to remove a portion of an epiretinal membrane, e.g., the surgeon may maneuver abrasive portion 1320 over the epiretinal membrane. In one or more embodiments, the surgeon may remove additional portions of the epiretinal membrane, e.g., by manipulating flexible tip 1305. Illustratively, the surgeon may manipulate flexible tip 1305 to raise a portion of an epiretinal membrane, e.g., the surgeon may maneuver abrasive portion 1320 over the epiretinal membrane to raise the portion of the epiretinal membrane. In one or more embodiments, a surgeon may grasp a raised portion of an epiretinal membrane, e.g., with a forceps, and peel the epiretinal membrane.

The foregoing description has been directed to particular embodiments of this invention. It will be apparent; however, that other variations and modifications may be made to the described embodiments, with the attainment of some or all of their advantages. Specifically, it should be noted that the principles of the present invention may be implemented in any system. Furthermore, while this description has been written in terms of a surgical instrument, the teachings of the present invention are equally suitable to any systems where the functionality may be employed. Therefore, it is the object of the appended claims to cover all such variations and modifications as come within the true spirit and scope of the invention.

What is claimed is:

1. A method comprising:
inserting a hypodermic tube of an instrument through an incision in an eye wherein the hypodermic tube has a hypodermic tube distal end and a hypodermic tube proximal end;
disposing the hypodermic tube distal end over a membrane;
squeezing a flow control mechanism of the instrument;
irrigating a visualization fluid out from a visualization fluid chamber of the flow control mechanism;
irrigating the visualization fluid through a visualization fluid conduit of a visualization fluid guide of the instrument;
irrigating the visualization fluid into the hypodermic tube proximal end;
irrigating the visualization fluid out from the hypodermic tube distal end;
irrigating the visualization fluid onto the membrane; and
staining the membrane with the visualization fluid.

2. The method of claim 1 wherein the visualization fluid is indocyanine green dye.

3. The method of claim 1 wherein the visualization fluid is kenalog.

4. The method of claim 1 wherein the visualization fluid is trypan blue dye.

5. The method of claim 1 wherein the visualization fluid guide is disposed in the flow control mechanism.

6. The method of claim 5 wherein an interface between a portion of the visualization fluid guide and a visualization fluid guide housing of the flow control mechanism comprises a hermetic seal.

7. The method of claim 1 further comprising:
decreasing a pressure within the flow control mechanism.

8. The method of claim 7 further comprising:
aspirating a portion of the portion of the visualization fluid into the hypodermic tube distal end.

9. The method of claim 7 further comprising:
exposing a portion of a pressure vent of the flow control mechanism.

10. The method of claim 1 wherein the visualization fluid flows out from the hypodermic tube distal end at a flow rate in a range of 0.1 to 10.0 milliliters per minute.

11. The method of claim 1 wherein the visualization fluid flows out from the hypodermic tube distal end in discrete volumes.

12. The method of claim 11 wherein the visualization fluid flows out from the hypodermic tube distal end in discrete drops having a volume in a range of 0.001 to 0.08 milliliters.

13. The method of claim 1 further comprising:
irrigating the visualization fluid out from a soft tip.

14. The method of claim 13 wherein the visualization fluid flows out from the soft tip at a flow rate in a range of 0.1 to 4.5 milliliters per minute.

15. The method of claim 1 further comprising:
irrigating the visualization fluid out from a flexible tip.

16. The method of claim 1 further comprising:
performing an air-fluid-exchange.

17. The method of claim 16 further comprising:
maintaining an intraocular pressure in the eye.

18. The method of claim 16 further comprising:
decreasing a pressure within the visualization fluid conduit.

19. The method of claim 18 further comprising:
aspirating a vitreous out from the eye.

20. The method of claim 19 further comprising:
forming a gas bubble in the eye.
